(19) United States
(12) Patent Application Publication
(10) Pub. No.: US 2020/0397751 A1
(43) Pub. Date: Dec. 24, 2020

(54) Modulation of AMPA/kainate Receptors for the Treatment of Hypoglycemia

(71) Applicant: Peter Vanderklish, San Diego, CA (US)

(72) Inventor: Peter Vanderklish, San Diego, CA (US)

(21) Appl. No.: 16/770,401

(22) PCT Filed: Dec. 4, 2018
(86) PCT No.: PCT/US18/63907

§ 371 Date: Jun. 5, 2020

Related U.S. Application Data
(60) Provisional application No. 62/594,866, filed on Dec. 5, 2017.

(51) Int. Cl. A61K 31/4015; A61K 9/00; A61P 3/10

(52) U.S. Cl. CPC A61K 31/4015; A61P 3/10; A61K 9/0053

(57) ABSTRACT

Methods for modulating the levels of glucagon and blood glucose of a mammal are provided. In the subject methods, a positive allosteric modulator of AMPA/kainate receptors is administered to a host. The subject methods find use in applications where it is desired to increase one or both of the glucagon and blood glucose levels in a mammalian host. The subject methods find use in applications where it is desired to decrease the size, or breadth, of the circadian range of blood glucose levels in a mammalian host. The subject methods also find use in applications where it is desired to decrease the frequency, severity, or occurrence of hypoglycemia in a mammalian host. Finally, the subject method finds use in applications where it is desired to decrease the frequency, severity, or occurrence of nocturnal hypoglycemia in a mammalian host, particularly that which occurs in diabetics as a result of therapy with insulins or insulin analogs or other glucose lowering agents, or combinations of such agents.

### CROSS-REFERENCE TO RELATED APPLICATION

Pursuant to 35 U.S.C. § 119(e), this application claims priority to the filing date of the U.S. Provisional Patent Application Ser. No. 62/594,866, filed Dec. 5, 2017, the disclosure of which application is herein incorporated by reference

### INTRODUCTION

Diabetes compromises a range of metabolic disorders that can be generally characterized by a host's inability to properly modulate their blood glucose levels. Typically, the art defines one major class of diabetes as “Type 1,” “insulin-dependent,” “juvenile diabetes.” and the like, which is generally characterized by insufficient production of insulin. A second major type of diabetes is typically termed “Type II” and is generally described as cellular “resistance” to insulin. This type of diabetes was once typically referred to as “adult-onset” diabetes, prior to the occurrence of higher rates of Type II diabetes in the adolescent population, as well as its growing occurrence with the obese of any age.

Over 26 million people have diabetes in the United States alone, representing one of the country's largest societal health care expenditures. If costs for both the management of diabetes ($174 billion) and the treatment of conditions it plays a major role in, such as heart disease and stroke ($432 billion), are taken together, the societal burden of the disease easily exceeds 500 billion dollars per year. Insulin therapy is utilized in all cases of Type I diabetes and in a sizeable percentage of Type II cases. Diabetics typically use insulin either as a monotherapy, or they combine insulin dosing with another oral antiglycemic agent.

A significant complication of insulin usage is that it can trigger hypoglycemia. Approximately 45% of all hypoglycemic episodes resulting from insulin usage occur at night. This “nocturnal hypoglycemia” is particularly problematic as it occurs when neural mechanisms involved in maintaining glucose homeostasis are blunted. Additionally, both cognitive awareness of hypoglycemic symptoms, and behavioral responses to them, are absent or impaired due to the subject's lack of consciousness.

Recent studies suggest that occult nocturnal hypoglycemia occurs 68% of the time in children with Type I diabetes, and that severe episodes are common. Even when nocturnal hypoglycemia is occult/asymptomatic, it can lead to deterioration of glycemic control during waking hours in insulin-treated diabetics. Type II diabetics under insulin therapy also encounter nocturnal hypoglycemia at a significant rate, as do subjects who use oral hypoglycemic agents such as metformin and sulfonylureas.

This frequent complication for those suffering from diabetes presents numerous acute and long-term health risks, such as increased inflammation, pro-atherogenic effects, and the like. This is particularly true for risks associated with the brain, due to its obligate use of glucose as an energy source. Neurological complications of hypoglycemia include confusion, ataxia, seizure, and a variety of other manifestations of impaired cerebral function.

The body's coping mechanism for hypoglycemia is multifold, and can include decreased secretion of insulin, as well as various neuroendocrine responses. A major homeostatic response is the secretion of glucagon, a peptide hormone that stimulates gluconeogenesis and glycogenolysis in the liver and a corresponding rapid increase in blood glucose levels. Glucagon is secreted from pancreatic alpha cells (α-cells) in response to low glucose, by mechanisms involving local sensing of arterial glucose and both sympathetic and parasympathetic inputs. Glucagon responses are blunted in the elderly, making this largely Type II population even more susceptible to nocturnal hypoglycemia. In Type I diabetics, the threshold for induction of glucagon secretion can vary as a function of long-term insulin use.

Current strategies to avoid nocturnal hypoglycemia include the use of long acting insulins designed to exhibit stable activity over time. They also include the common practice of eating carbohydrate-rich foods prior to bedtime, a behavioral strategy that ends up leading to weight gain, which, in turn, can exacerbate Type II diabetes.

Accordingly, there is a large unmet need for new methods and compounds to counteract, lessen, or prevent hypoglycemic events, especially during the nighttime hours, and especially in subjects suffering from diabetes, pre-diabetes, and related blood glucose disorders, and particularly for subjects under treatment with insulin, insulin analogs, other glycemic control agents, or a mixture of such medications.

Additionally, there is an unmet medical need for new methods and compounds that can counteract hypoglycemia arising from prescription drug usage and other metabolic disorders, and to prevent large swings in blood glucose levels in a host as well as large ranges of circadian blood glucose levels. Ideally, the methods and compounds would augment the body's natural homeostatic mechanisms for maintaining euglycemia without inducing glucose spikes on their own.

A key variable in the mechanism of glucagon release from pancreatic alpha-cells is the membrane potential of such cells. In the case of hypoglycemia, we have already discussed that the use of positive allosteric modulators of AMPA/kainate receptors can be used to enhance glutamate-mediated depolarization of pancreatic alpha-cells during hypoglycemia, thereby potentiating glucagon release and enhancing a subject's homeostatic response to low glucose.

The importance of alpha-cell polarization state in glucagon regulation is underscored by the fact that another type of ionotropic receptor exerts an opposing (inhibitory) effect on glucagon secretion. Specifically, chloride-conducting GABA-A receptors (GABA-Rs) hyperpolarize alpha-cells and thereby inhibit the downstream release of glucagon. GABA-Rs are typically activated by GABA released from beta-cells under conditions of increased glucose (such as during the post-prandial period). Insulin release coincident with GABA from beta-cells has the effect of enhancing membrane expression of GABA-Rs and their response to ligand through a mechanism that typically involve the activation of the intracellular kinase Akt. In subjects without diabetes, this mechanism mediates the post-prandial shutdown of glucagon release, thus allowing insulin to buffer prandial glucose spikes without added glucose from the liver.

Typically, in subject with Type II diabetes, GABAergic regulatory mechanisms are impaired at two levels. First, Type II diabetic subject often have a diminished early phase (i.e. immediate post-prandial) insulin spike. Their late phase insulin release, however, remains mostly intact, but is typically insufficient to normalize glucose to baseline levels before the next meal. Second, there is insulin resistance at the level of insulin receptor (IR) signaling. These deficits can lead to reduced GABA-R activation in alpha-cells in response to release of GABA from beta-cells. The result of this is that alpha-cells can become more depolarized than they would normally be during a prandial glucose spike, and at later inter-meal phases. A state of increased glucagon levels during the daytime period is thus observed in a majority of Type II diabetics.

Accordingly, there is a large unmet need for new methods and compounds to counteract, lessen, or prevent increased glucagon levels, especially during the during the daytime period, and especially in subjects suffering from diabetes, pre-diabetes, and related blood glucose disorders, and particularly for subjects under treatment with insulin, insulin analogs, other glycemic control agents, or a mixture of such medications.

Accordingly, there is a large unmet need for new methods and compounds to counteract, lessen, or prevent hyperglycemia in Type II diabetic, especially during the during the daytime period.

### SUMMARY

Methods for modulating mammalian hypoglycemia are provided. In the subject methods, allosteric modulators of AMPA/kainate receptors are administered to a subject.

The subject methods find use in the treatment of nocturnal hypoglycemia in a host.

The subject methods find use in the treatment of nocturnal hypoglycemia associated with diabetes in a host.

The subject methods find use in a variety of different applications where modulation of blood glucose levels and/or modulation of hypoglycemia in a mammal is desired, such as in the treatment of glycemic dysfunction, particularly the occurrence of, or increased magnitude, severity, or frequency of, hypoglycemic episodes as typically occurs in diabetics under therapy with insulin, and/or insulin analogs, and/or hypoglycemic agents.

They subject methods also find use in treatment of hypoglycemia that is the result of one or more metabolic disorders.

They subject methods also find use in treatment of hypoglycemia that is idiopathic.

They subject methods also find use in treatment of hypoglycemia that is the result of a side effect of a medication.

The subject methods also find use in a variety of different applications where a host is in need of a smaller circadian range of blood glucose levels.

The methods and compounds described here relate to the ability of positive modulators of AMPA/kainate receptors to treat a host in order to enhance glucagon secretion from the pancreas. The methods and compounds described also relate to compounds capable of interacting with AMPA/kainate receptors, allosterically or otherwise, in such a way as to enhance glucagon secretion in a host.

Ideally, when blood glucose levels are low in a host, glucagon is released from pancreatic alpha-cells. This homeostatic response is critically dependent on, and greatly potentiated by, an autocrine step in which glutamate is released from pancreatic alpha-cells, then binds to AMPA/kainate class glutamate receptors on the same cells to cause depolarization, calcium entry, and amplification of the exocytotic release of glucagon.

In a host suffering from hypoglycemia, however, these processes may be inhibited, malfunctioning, or absent, or they may be intact, but acting at a level insufficient to counteract hypoglycemia associated with the use of insulin, and/or insulin analogs, and/or hypoglycemic agents. In a diabetic under therapy with insulin, and/or insulin analogs, and/or antiglycemic agents (e.g. Metformin, sulfonylureas, and the like), the autocrine mechanism step mentioned above can, by the practice of the instant invention, be pharmacologically potentiated to offset hypoglycemic episodes, particularly nocturnal hypoglycemic episodes.

The methods described here find use in providing a host with a compound capable of exerting positive allosteric modulation of an AMPA/kainate receptor in order to potentiate glutamate-induced cellular depolarization within alpha-cells of the pancreas of a host during hypoglycemia, and thereby provide glucagon for a host in need of increased glucagon activity, such as during hypoglycemic episodes, and particularly in nocturnal hypoglycemia episode that occur in a host with diabetes.

The instant invention discloses that any compound capable of positive allosteric modulation of AMPA/kainate receptors can be used as an effective means for modulating, lessening, or preventing hypoglycemia in a host, particularly nocturnal hypoglycemia, and more particularly nocturnal hypoglycemia arising from the treatment of diabetes or from other causal mechanisms.

The disclosed compounds and compound classes, mechanisms of action, and methods can also be thought of as enabling a nightly “insurance policy” to, optionally, accompany the use of insulin, and/or insulin analogs, and/or other antiglycemic compounds or devices.

It is an object of the invention to treat hypoglycemia in a host.

It is an object of the invention to provide a therapeutic regime for treating low levels of blood glucose in a host.

It is an object of the invention to provide a therapeutic regime for treating hypoglycemia that occurs at night in diabetics treated with insulins, and/or insulin analogs, and/or other glucose lowering medications or devices.

It is an object of the invention to provide a therapeutic regime for providing a narrower circadian range of blood glucose levels in a host.

An advantage of the invention is that a host's endogenous mechanisms for glucagon release are those that are modulated.

An advantage of the invention is that said compounds, acting allosterically on AMPA/kainate receptors, would not induce significant glucose spikes on their own during euglycemia (when glutamate is not released), but would act to enhance a host's homeostatic glucagon response when hypoglycemia occurs.

A feature of the invention is that formulations with specific positive modulators of AMPA/kainate type glutamate receptors are employed.

A feature of the invention is that formulations with specific positive modulators of AMPA/kainate type glutamate receptors (that do not cross the blood-brain-barrier) may be employed.

A feature of the invention is that formulations with specific positive modulators of AMPA/kainate type glutamate receptors may be employed according to certain temporal patterns.

A feature of the invention is that formulations with specific positive modulators of AMPA/kainate type glutamate receptors are employed in the time period prior to when a subject goes to sleep for the night.

Methods for modulating mammalian glucagon levels are provided. In the subject methods, direct antagonists or negative allosteric modulators of AMPA/kainate receptors are administered to a subject.

The subject methods find use in the treatment of excessive daytime glucagon levels in a host.

The subject methods find use in a variety of different applications where modulation of glucagon levels and/or modulation of hyperglycemia in a mammal is desired.

They subject methods also find use in treatment of high glucagon level that is the result of one or more metabolic disorders.

The methods and compounds described here relate to the ability of antagonists of AMPA/kainate receptors to treat a host in order to decrease glucagon secretion from the pancreas. The methods and compounds described also relate to compounds capable of interacting with AMPA/kainate receptors, allosterically or otherwise, in such a way as to decrease glucagon secretion in a host.

The methods described here find use in providing a host with a compound capable of exerting direct antagonistic or negative allosteric modulation of an AMPA/kainate receptor in order to inhibit glutamate-induced cellular depolarization within □ alpha-cells of the pancreas of a host during hyperglycemia, and thereby inhibit glucagon release in a host in need of decreased glucagon activity, such as during hyperglycemic episodes, and particularly in daytime hyperglycemia episode that occur in a host with Type II diabetes.

The instant invention discloses that any compound capable of direct antagonistic or negative allosteric modulation of AMPA/kainate receptors can be used as an effective means for modulating, lessening, or preventing hyperglycemia in a host, particularly daytime hyperglycemia, and more particularly daytime hyperglycemia arising in a subject suffering from Type II diabetes.

It is an object of the invention to treat hyperglycemia in a host.

It is an object of the invention to provide a therapeutic regime for treating high levels of blood glucagon in a host.

A feature of the invention is that formulations with specific antagonists or negative modulators of AMPA/kainate type glutamate receptors are employed.

A feature of the invention is that formulations with specific antagonists or negative modulators of AMPA/kainate type glutamate receptors that do not cross the blood-brain-barrier may be employed.

A feature of the invention is that formulations with specific antagonists or negative modulators of AMPA/kainate type glutamate receptors that do cross the blood-brain-barrier may be employed.

### BRIEF DESCRIPTION OF THE FIGURES

FIG. 1A-1B shows a schematic depicting the different roles of alpha and beta cells of the pancreas, and how such cells typically react to changing blood glucose levels in a host. FIG. 1A depicts how the two different cells react to low blood glucose levels. Under conditions of low glucose, alpha-cells typically release the peptide hormone glucagon, which acts to increase systemic glucose levels by acting on the liver to enhance gluconeogenesis, glucose release, and glycogenolysis. The release of glucagon from alpha cells involves an autocrine step in which the amino acid/neurotransmitter glutamate is released from the alpha-cells and binds to glutamate receptors of the AMPA/kainate class to amplify glucagon secretion (see FIG. 2 for greater detail). FIG. 1B depicts how the two cell types react to high blood glucose concentrations Typically, under conditions of high glucose, beta-cells release insulin, which is involved in the lowering of blood glucose levels and the uptake of glucose in target tissues.

FIG. 2A-2B show a schematic depicting a mechanism of action for certain glucagon secretion steps of the present disclosure. FIG. 2A depicts the typical process of glucagon secretion. Typically, under conditions of low glucose, such during the nighttime hours in subjects being treated with insulins, or insulin analogs, or glucose lowering agents, and the like, glutamate is released from alpha-cells. Released glutamate binds to glutamate receptors of the AMPA/kainate class that induce cellular depolarization. Depolarization, in turn, leads to the activation of voltage gated calcium channels, which admit calcium into the cell that, in turn, enhances the release of glucagon from granules. These granules also contain glutamate, which can further amplify glucagon secretion by the aforementioned autocrine mechanism. FIG. 2B depicts enhanced glucagon secretion by practicing exemplary methods of the present disclosure. The subject methods affect the autocrine step to enhance the depolarization elicited by glutamate and mediated by AMPA/kainate receptors. This leads to greater depolarization, increased voltage gated calcium channel activity and calcium entry, and therefore the enhance release of glucagon. In enhancing an autocrine step that is triggered initially by low glucose, the method is superior to standard options/strategies for combating hypoglycemia since it does not raise glucagon and blood glucose levels unless the subject has entered a hypoglycemic state; i.e. the methods and compounds described do not trigger glucose spikes on their own, but act as an “insurance policy” to offset hypoglycemia if it does occur.

FIG. 3A-3B shows various exemplary compounds within the class of positive allosteric modulators of AMPA/kainite receptors that are capable of positively modulating AMPA/kainate receptors. FIG. 3A shows exemplary compounds of interest derived from piracetam. FIG. 3B shows exemplary compounds of interest including examples of benzamides, benzothiadiazines, biaryl propylsulfonamides and trifluoromethylpyrazoles. Some of the species shown can be optionally chemically modified to limit their ability to pass through the blood brain barrier.

FIG. 4A-4C shows a schematic depicting how the treatment of a subject with an AMPA/kainate receptor antagonist or negative modulator would affect GABA receptors and the regulation of glucagon levels in the subject. FIG. 4A depicts the mechanisms regulating glucagon release in normal individuals. During prandial glucose spikes. GABA-Rs on β-cells are activated by GABA released from β-cells; the responsivity of GABA-Rs to released GABA is greatly enhanced by insulin receptor (IR) signaling through the kinase Akt triggered by coincident release of insulin. GABA-R activation leads to hyperpolarization, which inhibits glucagon secretory mechanisms that are based on β-cell depolarization and subsequent calcium entry through VGCCs. Reduced glucagon release ensures that prandial glucose is not added to by glucose from the liver. FIG. 4B depicts how GABAergic mechanism for suppressing glucagon release via membrane hyperpolarization is impaired in In Type II diabetics. A reduced early phase prandial insulin spike, and insulin resistance are depicted. Glucagon release is thus not shut down and adds to post-prandial glucose elevations. FIG. 4C depicts how the deficit in alpha-cell GABA receptor mediated hyperpolarization during glucose spikes in Type II diabetics can be offset by reducing the activity of AMPA/kainate receptors, either through the use of direct antagonists or negative allosteric modulators.

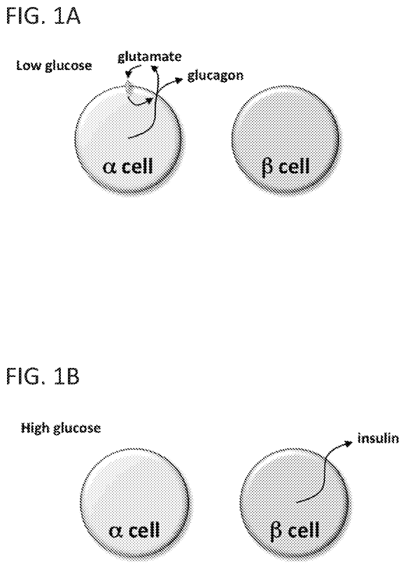

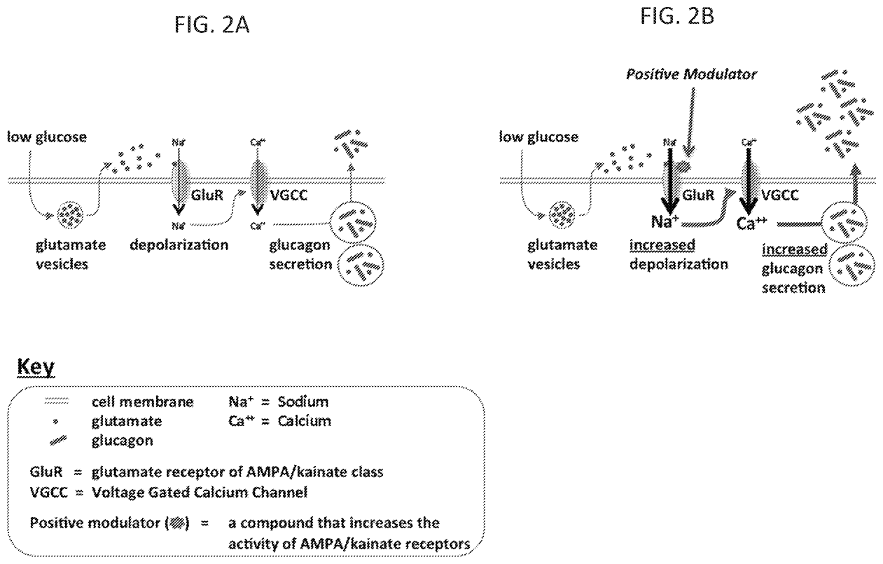

### DETAILED DESCRIPTION

Methods of modulating blood glucose levels of a mammalian host are provided. In the subject methods, a therapeutically effective amount of a compound capable of binding to an AMPA/kainate type glutamate receptor is administered to a mammalian host. In the subject methods, a therapeutically effective amount of a compound capable of increasing the activity of an AMPA/kainate type glutamate receptor is administered to a mammalian host.

AMPA (α-amino-3-hydroxy-5-methyl-4-isoxazolepropionic acid) is a compound that is a specific agonist for the AMPA receptor, where it mimics the effects of the neurotransmitter glutamate. Kainate receptors, or kainic acid receptors (KARs), are ionotropic receptors that respond to the neurotransmitter glutamate.

The subject methods find use in a variety of applications where regulation of the blood glucose level of mammalian is desired, such as in the treatment of undesired low blood glucose levels, the modification of large, undesired, circadian ranges in blood glucose concentrations, the treatment of hypoglycemia, the treatment of hypoglycemia caused by prescription drug use, the treatment of idiopathic hypoglycemia, the treatment of nocturnal hypoglycemia, and especially the treatment of nocturnal hypoglycemia in a host with diabetes or other related metabolic disorder.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The provide dates of publication may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In the subject methods, a compound capable of binding to an AMPA/kainate receptor is administered to a mammalian host to modulate glucose levels in a mammalian host. Compounds suitable for use in the subject methods are generally those that amplify (up-regulate, up-modulate. “positively modulate.” increase, supplement, and the like) the activity of AMPA/kainate receptors, in some cases through allosteric modulation.

Compounds suitable for such use may be identified by any convenient methods, such as the assay systems described in U.S. Pat. No. 6,620,808, and are hereby incorporated by reference.

In the subject methods, compounds described here can augment glucagon secretion from pancreatic alpha-cells, and potentially other cells that may be capable of secreting glucagon. Such compounds up-regulate glucagon release. Typically, when blood glucose levels drop, pancreatic alpha-cells release the amino acid glutamate that then binds to a class of ionotropic glutamate receptors known as AMPA/kainate receptors. This glutamate binding leads to cellular depolarization that, in turn, triggers the secretion of glucagon.

The instant invention details a method that includes the application and utilization of any members of the broad class of structurally varied compounds known as positive allosteric modulators of AMPA/kainate receptors. The instant invention details how positive allosteric modulators of AMPA/kainate receptors can be used to treat a host in order to enhance the aforementioned glutamate-driven step of glucagon secretion. Since such positive modulator compounds bind in an allosteric fashion, and therefore do not typically activate AMPA/kainate receptors on their own (i.e. without co-binding of the ligand glutamate), they typically cannot directly (i.e. autonomously) elicit glucagon release. However, when glucose is low and glutamate is released from alpha-cells, positive allosteric modulators of AMPA/kainate receptors will potentiate the cellular depolarization(s) induced by the binding of glutamate to AMPA/kainate receptors on alpha-cells, and thereby provide needed glucagon release(s) to a host.

The action of a compound can be verified by measurement of the responses it induces via AMPA/kainate receptors (for one such measurement method see Staubli et al., 1994). Time courses of distribution can be ascertained via oral or injection delivery and subsequent quantitation of compound levels can be taken in various tissue and blood samples. Quantitation can be accomplished by methods known to those skilled in the art and will vary depending on the chemical and biologic nature of the compound and testing system.

Compounds useful in the practice of this invention are generally those that amplify the activity of endogenous AMPA/kainate receptors, particularly by amplifying the conductance, reducing the desensitization, altering ligand binding, or otherwise modifying the kinetic and/or pharmacological, or other, properties of the receptors to achieve enhanced depolarization.

As a group, positive allosteric modulators of AMPA/kainate receptors are quite varied in structure. For a compound to be used in the practice of this invention, structure is irrelevant. As long as a given compound acts as a positive modulator of AMPA/kainate receptors it will work in the practice of this invention.

Compounds of interest include, but are not limited to, compounds identifiable by the assays described above. Structural families within the class of positive allosteric modulators of AMPA/kainate receptors include, but are not limited to: AMPAkines, benzamides, pyrrolidines, benzoylpiperidines, benzoylpyrrolidines, benzothiazides, benzothiadiazines, biarylpropylsulfonamides, benzylperazines, pyrazole amide derivatives, trifluoromethylpyrazoles and the like.

The instant invention discloses that any compound capable of positive allosteric modulation of AMPA/kainate receptors can be used as an effective means for modulating, lessening, or preventing hypoglycemia, particularly, but not limited to, nocturnal hypoglycemia associated with a diabetes treatment, in a host. The disclosed compounds and compound classes, mechanisms of action, and methods can be thought of as enabling a nightly “insurance policy” to, optionally, accompany the use of insulin, and/or insulin analogs, and/or the use of these therapeutics with other antiglycemic compounds or devices.

The instant invention also discloses that any compound capable of positive allosteric modulation of AMPA/kainate receptors can be used as an effective means for lessening, or providing better control of, the circadian range of blood glucose levels in a host at any time of day. This includes, but is not limited to, use of the methods and compounds in the context of nocturnal hypoglycemia in diabetics to achieve better glycemic control during waking hours.

The instant invention also discloses that any compound capable of positive allosteric modulation of AMPA/kainate receptors can be used as an effective means for lessening, preventing, or providing better control of idiopathic hypoglycemia.

The instant invention also discloses that any compound capable of positive allosteric modulation of AMPA/kainate receptors can be used as an effective means for lessening, preventing, or providing better control of hypoglycemia associated with the use of prescription drugs.

Foremost among the compounds which can be used in the practice of the invention is a broad class of compounds, typically known as “ampakines,” that had their chemical origins in part as structural derivatives of the nootropic drug aniracetam. The instant invention describes the use of any positive allosteric modulator of AMPA/kainate receptors, and, in particular, agents selected from the ampakine class of compounds, for treating a host in need, particularly a host suffering from bouts of hypoglycemia, and more particularly to a host that has bouts of nocturnal hypoglycemia associated with diabetes therapy, such as insulins, and/or insulin analogs, and/or hypoglycemic agents, or the combined use of any of these agents.

The invention similarly teaches the use of cyclothiazide, derivatives of cyclothiazide, other classes of positive allosteric modulators of AMPA/kainate receptors, and commonly used nootropics such as piracetam, aniracetam, and related compounds, all of which share the common mechanism of action (positive allosteric modulation of AMPA/kainate receptors) to be used for treating a host who has, or is pre-disposed to, episodes of hypoglycemia (such occurs nocturnally in diabetics undergoing any of the aforementioned treatments) or undesirably large circadian ranges in blood glucose levels.

Additionally, the invention details the use of variants within the class of positive allosteric modulators of AMPA/kainate receptors that have been modified to (or display properties consistent with a propensity to) lessen, or block, their ability to cross the blood brain barrier, as treatments for hypoglycemia, particularly nocturnal hypoglycemia, and more particularly nocturnal hypoglycemia associated with diabetes, insulin therapy, oral hypoglycemic agents or their combined use, as well as large circadian ranges in blood glucose levels and other related disorders.

Turning now to the subject methods, a therapeutically effective amount of one or more, usually no more than 5, more usually no more than 2, types of distinct allosteric modulators of AMPA/kainate receptors, as described above, are administered to a mammalian host in which modulation of blood glucose levels is desired. The term “therapeutically effective amount” means an amount effective to cause a modulation or alteration in the blood glucose levels of the host being treated, usually by changing the levels of glucagon in the host.

The compounds of this invention can be incorporated into a variety of formulations for therapeutic administration, i.e. combined with a physiological acceptable vehicle to produce a pharmaceutical composition. Examples of suitable pharmaceutical compositions include capsules, tablets, syrups, suppositories, gel, patches and various injectable forms. Administration of the compounds can be achieved in various ways, including oral, bucal, rectal, parenteral, intraperitoneal, intradermal, transdermal administration. Formulations of particular interest of the compounds are oral preparations, particularly capsules or tablets.

Dose levels can vary as a function of the specific compound, the severity of the symptoms, and the susceptibility of the subject to side effects Some of the specific compounds that increase the activity, drive, or responsiveness of AMPA/kainate receptors are more potent than others. Preferred dosages for a given compound are readily determinable by those of skill in the art by a variety of means. A preferred means is to measure the physiological potency of a given compound that is a candidate for administration, and such methods are discussed in U.S. Pat. No. 6,620,808, and are hereby incorporated by reference.

The above described compounds and/or compositions are administered at a dosage sufficient to achieve the desired modulation of blood glucose levels while minimizing side effects. Typical dosages for systemic administration range from 0.1 to 10 milligrams per kg weight of subject per administration, but may vary outside this range depending on the potency, metabolism, and tissue distribution of the particular compound

The dosing may be scheduled as desired, where a dose is administered nightly before a subject goes to bed, or once before a subject goes to sleep and then once more in the middle of the night, or periodically over a given period of time (e.g. once a day for a month or longer, twice a day on a daily basis for two weeks) and the like. In certain instances, the dosing may be performed intermittently, e.g., at irregular intervals as needed. In certain cases, such dosing may be preferred in a host having diabetes when a calculated nighttime insulin dosage is high.

For example, a typical dosage may be one 10-50 mg tablet taken once a day, or as a time-release capsule, or tablet, taken prior to when a subject goes to sleep for the night. The time-release effect may be obtained by capsule, or tablet, materials that dissolve at different pH values, by capsules, or tablets, that release slowly by osmotic pressure, or by any other known means of controlled release. Some compounds may exhibit pharmacokinetic properties that are optimal for nighttime hypoglycemic control without the need for controlled release; these may be delivered as rapidly dissolving pills or capsules, tablets, liquids, or any of the aforementioned compositions or routes that afford rapid assimilation of the compound.

In addition, the present invention provides for kits with unit doses of one of more compounds or dosages capable of increasing the activity of AMPA/kainate receptors in oral, injectable, or other routes of administration. In addition to the containers containing the unit doses will be an informational package insert describing the use and attendant benefits of the compounds in treating blood glucose levels, hypoglycemia, nocturnal hypoglycemia, diabetes treatment-related hypoglycemia, modulating the range of circadian blood glucose levels, and the like. Preferred compounds and unit doses include those described herein above.

The subject methods may be used to modulate the blood glucose levels, or blood glucagon levels, of a variety of different mammalian hosts. Mammalian hosts which may be treated according to the subject methods include humans and rare and/or valuable animals, domestic animals, such as livestock, e.g. horses, cows, pigs, sheep, and the like; and pets, e.g. dogs, cats, and the like.

By “modulation of blood glucose levels” it is meant that the concentration of glucose in a host's blood or body fluids is altered in some manner, usually through a modulation or change in the level of glucagon release from pancreatic alpha cells and blood circulatory levels of glucagon, where modulation includes increasing the secretion of glucagon, usually increasing the blood or fluid level of glucose in the host, in response to the administration of the compound capable of increasing the activity of an AMPA/kainate receptor.

In addition to the treatment of hypoglycemia, the treatment of nocturnal hypoglycemia, the treatment of diabetes medication-related hypoglycemia, the treatment of nocturnal hypoglycemia associated with diabetes medications, the treatment of idiopathic hypoglycemia, the treatment of hypoglycemia associated with prescription drug use, and the modulation of large circadian swings in glucose, the subject methods find use in a variety of diverse applications where one wishes to modulate blood glucose levels and/or glucagon levels in a mammalian host.

Representative applications in which the subject methods find use include the treatment of symptoms or diseases associated with, or resulting from, the dysfunction of a host's endogenous regulation of his or her blood glucose levels, where dysfunction can also refer to the hyposecretion or hypoactivity of glucagon and/or hypoglycemia.

The terms “treatment,” “treating,” and “treat” and the like are used herein to generally mean obtaining a desired pharmacology and/or physiologic effect. The effect may be prophylactic in terms of completely or partially preventing a disease or symptom thereof and/or may be therapeutic in terms of a partial or complete cure for a symptom, disease, and/or adverse effect attributable to the disease. “Treatment” therefore includes: (a) preventing the disease or symptom from occurring in a subject which may be predisposed to the disease or symptom but has not yet been diagnosed as having the disease or symptom; (b) inhibiting the symptom or disease, i.e. arresting its development; or (c) relieving the symptom or disease, i.e. causing regression of the disease or symptom.

Of particular interest is use of the subject methods and compounds to up-regulate the secretion of glucagon. Thus, by administering a pharmaceutical composition comprising a compound capable of up-regulating the activity of AMPA/kainate receptors in a host, one up-regulates the production of glucagon in a host that is in a hypoglycemic state, which in turn increases the circulatory levels of glucose in the host.

Accordingly, one class of diseases or disorders which may be treated according to the subject methods are diseases or disorders associated with the loss of, or impairment in, effective blood glucose regulation, resulting in abnormally high and low circulatory levels of glucose in the mammal. By “treating” it is meant that the subject methods result in improved blood glucose regulation when compared to the level prior to treatment.

One “disease” characterized by such impairment in blood glucose regulation is diabetes and diabetes treatment-induced hypoglycemia. Therefore, the subject methods find use in the treatment of symptoms associated with diabetes, such as hypoglycemia that can arise nocturnally with the use of insulins, and/or insulin analogs and/or hypoglycemic agents, or combinations thereof. The subject methods also find use in treating hosts that have large ranges in their circadian blood glucose levels, and the like. Other diseases or disorders associated with impairment in blood glucose regulation may also be treated by the subject methods.

In certain embodiments, the methods include a step of diagnosing the need for treatment in a patient. As such, the methods include a step of determining that a patient is in need of treatment according to the invention. In certain embodiments, the methods are performed on patients that have been diagnosed or determined to be in need, or who are likely to be in need, of the treatment, especially diabetics using insulins and/or insulin analogs over the night time period, the practice of which can trigger episodes of hypoglycemia. Insofar as the methods described do not elicit significant glucose spikes when such patients are not in a hypoglycemic state, they can be thought of as constituting an “insurance policy” against the high likelihood of nocturnal hypoglycemic swings in such patients.

In other embodiments, the methods are performed on patients that have not been diagnosed, or determined to be in need of the treatment, but have a history of, or pre-disposition to have, pre-diabetes, diabetes, or other related diseases or disorders that comprise impaired control or regulation of blood glucose levels.

Methods of modulating blood glucagon levels of a mammalian host are provided.

In the subject methods, a therapeutically effective amount of a compound capable of inhibiting the activity of an AMPA/kainate type glutamate receptor is administered to a mammalian host.

The subject methods find use in a variety of applications where regulation of the blood glucagon level of mammalian is desired, such as in the treatment of undesired high blood glucose levels, the treatment of daytime hyperglycemia, and especially the treatment of daytime hyperglycemia in a host with Type II diabetes or other related metabolic disorder.

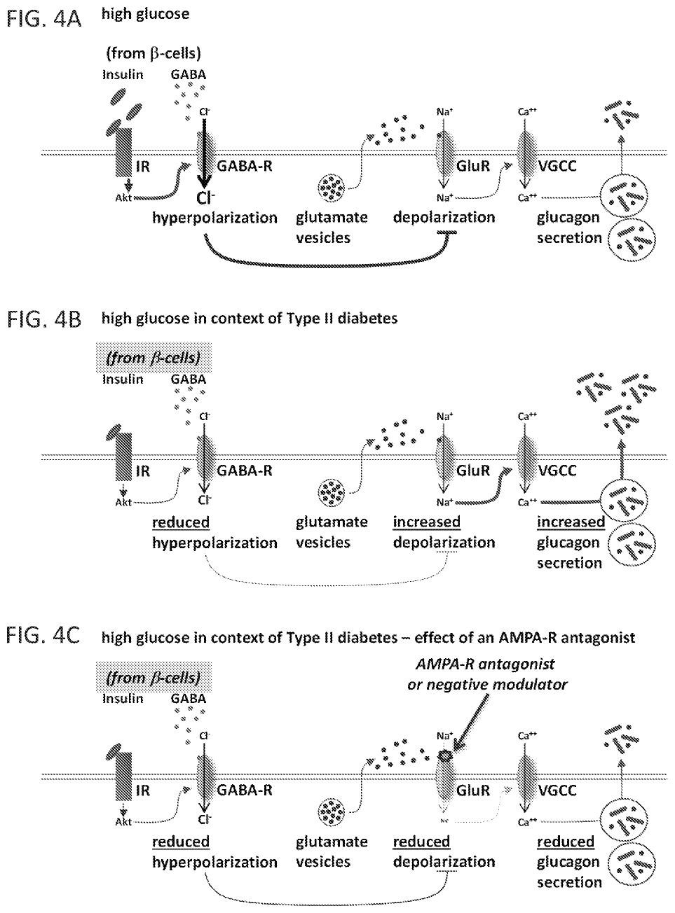

We propose that pharmacological reduction of AMPA/kainate receptor activity, either with direct antagonists or negative allosteric modulators, can be used to attenuate glucagon release and help normalize hyperglycemia in Type II diabetics. Compounds capable of acting in this manner (blocking depolarization) could effectively offset deficits in GABA-receptor-mediated hyperpolarization in alpha-cells. A schematic description of this method is shown in FIG. 4.

Such inhibitory AMPA/kainate receptor drugs can optionally be used as a companion product to the instant excitatory AMPA/kainate methods that find use in combating nocturnal hypoglycemia.

In one embodiment, negative/inhibitory AMPA/kainate receptor drugs can be used to normalize glucagon levels throughout the diurnal period and to reduce early post-prandial and steady state glucagon levels, while positive/excitatory modulators of AMPA/kainate receptors can be used at night to offset hypoglycemia in a subject.

Any convenient compounds that are capable of positively modulating AMPA/kainate receptors can find use in certain embodiments of the subject methods. Non-limiting examples of chemical structures of compounds within the class of positive allosteric modulators of AMPA/kainite receptors that find use in certain embodiments of the subject methods are set forth below. Some of the species shown can be optionally chemically modified to limit their ability to pass through the blood brain barrier.

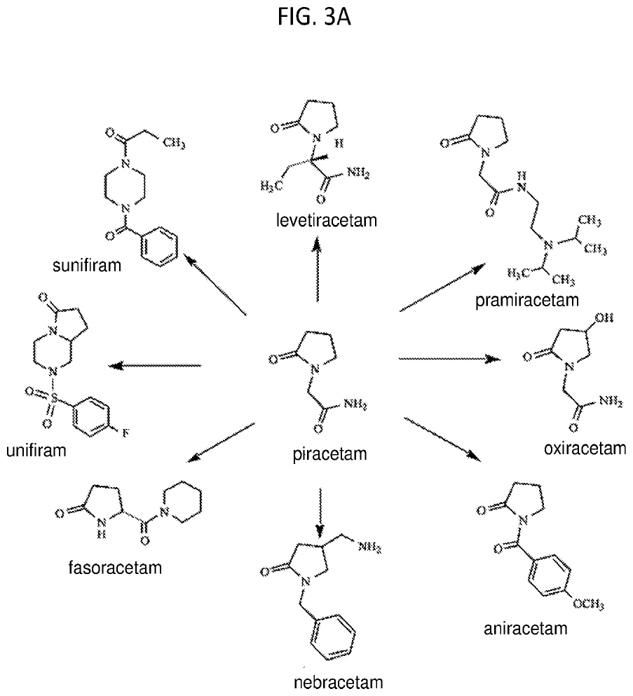

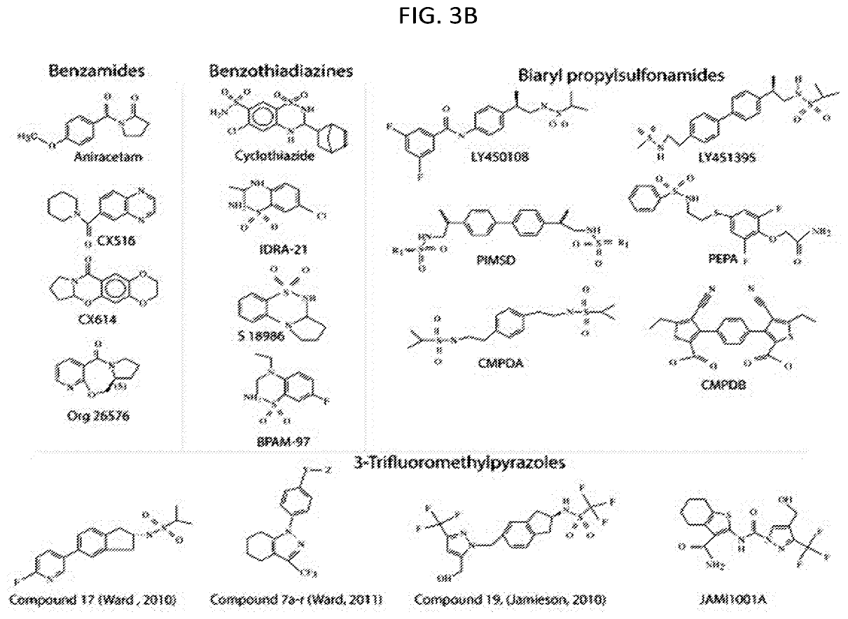

In some embodiments, a positive allosteric modulator of AMPA/kainite receptors is a compound of the class of racetams (e.g., compounds sharing a pyrrolidinone core structure). AMPAkines, benzamides, benzothiadiazines, biaryl propylsulfonamides or trifluoromethylpyrazoles. FIG. 3B shows exemplary compounds of these classes of positive allosteric modulators of AMPA/kainite receptors. FIG. 3A shows exemplary compounds of interest derived from piracetam (also known as 2-oxo-1-pyrrolidine acetamide). Classes and examples of AMPA receptor positive modulators of interest which find use in certain embodiments of the subject methods include those described by Reuillon et al. (2016: Curr Top Med Chem. 2016; 16(29):3536-3565). Partin (2014: Curr Opin Pharmacol. 2015 February; 20:46-53) and Larsen et al (2017: Mol Pharmacol. 2017 June; 91(6):576-585), the disclosures of which are herein incorporated by reference. Compounds which may be used or adapted for use as positive allosteric modulators of AMPA/kainite receptors in the subject compositions and methods include those compounds described in WO2010054336, U.S. Pat. Nos. 10,100,045, 9,328,125, 8,173,820, 7,504,390, 7,476,668, 7,393,868, 7,652,064, 7,625,932, 7,235,548, 6,730,677, 6,525,045 the disclosures of which are herein incorporated by reference.

Exemplary positive allosteric modulator of AMPA/kainite receptors compounds of interest include, but are not limited to, aniracetam, CX509 (see e.g., Suppiramaniam et al. Synapse. 2001 May; 40(2):154-8), CX516. CX614, Org26576, cyclothiazide (see e.g., Sharp et al., European Journal of Pharmacology: Molecular Pharmacology, 266. (1), 1994, Pages R1-R2), IDRA-21, S18986, BPAM-97, LY450108, PIMSD, CMPDA, LY451395 (mibampator), PEPA, CMPDB, compound 17 (FIG. 3B), compound 19 (FIG. 3B), JAMI1001A (FIG. 3B), 8-cyclopropyl-3-[2-(3-fluorophenyl)ethyl]-7,8-dihydro-3H-[1,3]oxazino[6,5-g][1,2,3]benzotriazine-4,9-dione, N-[(2S)-5-(6-fluoro-3-pyridinyl)-2,3-dihydro-1H-inden-2-yl]-2-propanesulfonamide, LY404187, LY392098, LY450108, CX546, CX691, S-18986, GSK729327, PF-04958242, IDRA21, BPAM121, BPAM344 and BPAM521.

In certain instances, the positive allosteric modulator of AMPA/kainite receptors is a benzamide, see e.g., Arai et al. (“Benzamide-type AMPA receptor modulators form two subfamilies with distinct modes of action,” J Pharmacol Exp Ther. 2002 December; 303(3):1075-85). FIG. 3B shows exemplary benzamide compounds of interest.

In certain instances, the positive allosteric modulator of AMPA/kainite receptors is a benzothiadiazine, see e.g., Battisti et al. (“An unexpected reversal in the pharmacological stereoselectivity of benzothiadiazine AMPA positive allosteric modulators”, Med. Chem. Commun., 2016.7, 2410-2417). FIG. 3B shows exemplary benzothiadiazine compounds of interest.

In certain instances, the positive allosteric modulator of AMPA/kainite receptors is a biaryl propylsulfonamide, see e.g., Ryder et al. (“Pharmacological characterization of cGMP regulation by the biarylpropylsulfonamide class of positive, allosteric modulators of alpha-amino-3-hydroxy-5-methyl-4-isoxazolepropionic acid receptors,” J Pharmacol Exp Ther. 2006 October; 319(1):293-8). FIG. 3B shows exemplary biaryl propylsulfonamide compounds of interest.

In certain instances, the positive allosteric modulator of AMPA/kainite receptors is a trifluoromethylpyrazole, see e.g., Caldwell et al. (“Rational Design of a Novel AMPA Receptor Modulator through a Hybridization Approach”. ACS Med Chem Lett. 2015 Apr. 9; 6(4): 392-396). FIG. 3B shows exemplary trifluoromethylpyrazole compounds of interest.

In some instances, the positive allosteric modulator of AMPA/kainite receptors is a racetam, such as a compound selected from aniracetam, brivaracetam, cebaracetam, coluracetam, dimiracetam, doliracetam, dupracetam, etiracetam/levetiracetam, fasoracetam, imuracetam, methylphenylpiracetam, nebracetam, nefiracetam, omberacetam (noopept), oxiracetam, phenylpiracetam, phenylpiracetam hydrazide, piracetam, pramiracetam, rolipram, rolziracetam, sunifiram, unifiram and seletracetam. FIG. 3A shows the structures of various piracetam derivatives of interest. See e.g., Löscher W. Richter A (March 2000). “Piracetam and levetiracetam, two pyrrolidone derivatives, exert antidystonic activity in a hamster model of paroxysmal dystonia”. European Journal of Pharmacology. 391 (3): 251-4, the disclosure of which is herein incorporated by reference.

In certain embodiments, the positive allosteric modulator of AMPA/kainite receptors compound is selected from one of the following structures:

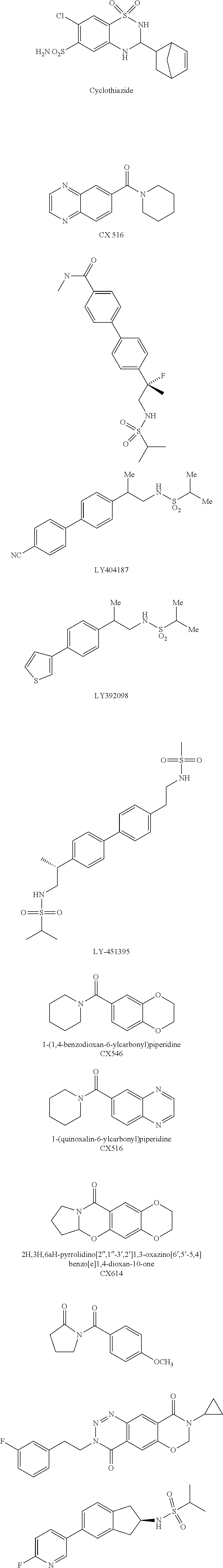

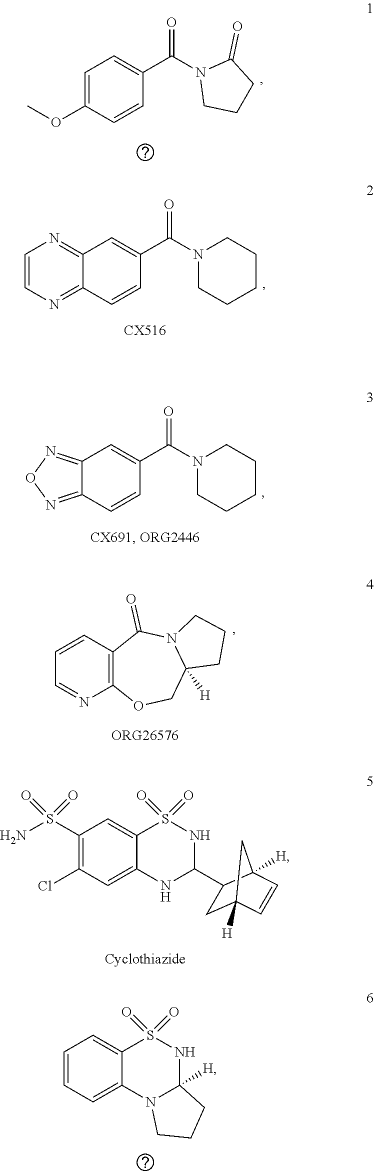

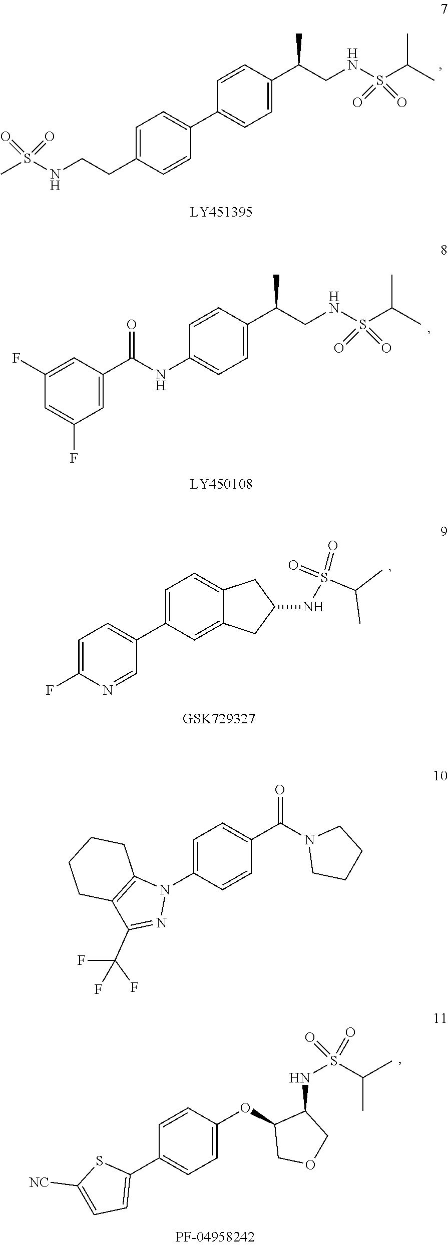 [SMILES: CC(C)S(=O)(=O)NC[C@H](C)c1ccc(-c2ccc(CCNS(C)(=O)=O)cc2)cc1 ; CC(C)S(=O)(=O)NC[C@H](C)c1ccc(NC(=O)c2cc(F)cc(F)c2)cc1 ; CC(C)S(=O)(=O)N[C@H]1Cc2ccc(-c3ccc(F)nc3)cc2C1 ; O=C(c1ccc(-n2nc(C(F)(F)F)c3c2CCCC3)cc1)N1CCCC1 ; CC(C)S(=O)(=O)N[C@H]1COC[C@H]1Oc1ccc(-c2ccc(C#N)s2)cc1]

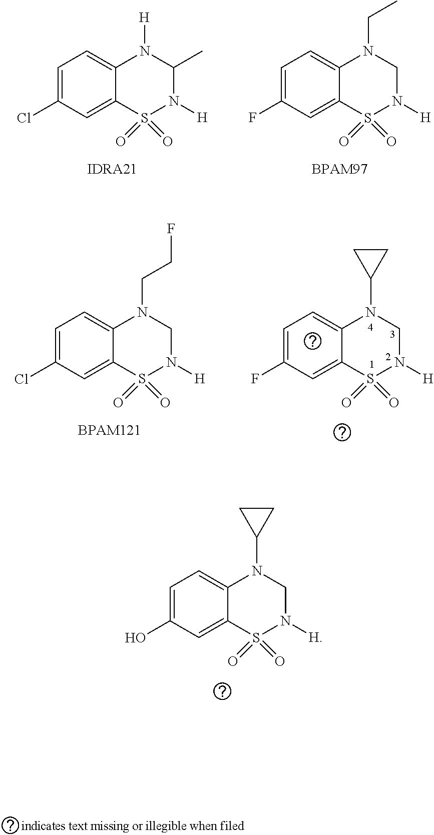

In certain embodiments, the positive allosteric modulator of AMPA/kainite receptors is a compound selected from one of the following structures:

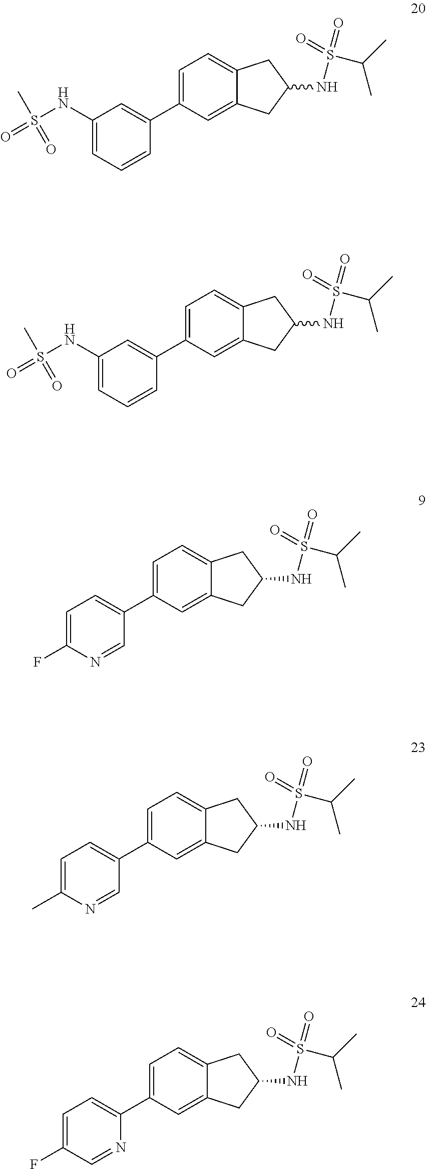

In certain embodiments, the positive allosteric modulator of AMPA/kainite receptors is compound 20 or a stereoisomer thereof:

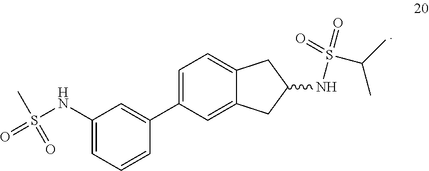 [SMILES: CC(C)S(=O)(=O)NC1Cc2ccc(-c3cccc(NS(C)(=O)=O)c3)cc2C1]

In some instances, the positive allosteric modulator of AMPA/kainite receptors is a 3-trifluoromethylpyrazole compound, see e.g., Ward et al. (“Integration of Lead Optimization with Crystallography for a Membrane-Bound Ion Channel Target: Discovery of a New Class of AMPA Receptor Positive Allosteric Modulators,” J. Med. Chem., 2011, 54(1), pp 78-94), the disclosure of which is herein incorporated by reference. FIG. 3 shows exemplary 3-trifluoromethylpyrazole compounds of interest, including general formula of compound 7a-r of Ward et al., 2011 (cited above), where in some cases, Y is alkyl (e.g., —CH₂—), substituted alkyl or —CO—, and Z is heterocycle or substituted heterocycle, such as pyrrolidine or pyrrolidinone. In certain cases, the 3-trifluoromethylpyrazole has one of the following structures:

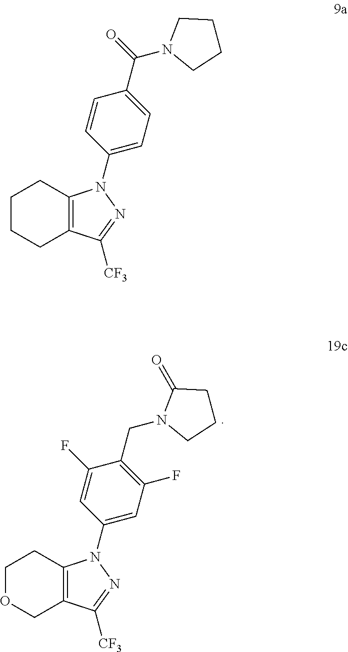 [SMILES: O=C(c1ccc(-n2nc(C(F)(F)F)c3c2CCCC3)cc1)N1CCCC1 ; O=C1CCCN1Cc1c(F)cc(-n2nc(C(F)(F)F)c3c2CCOC3)cc1F]

### Discussion

This application demonstrates that the facilitation of AMPA/kainate receptors can be used to: 1) increase the secretion of glucagon in a host, 2) normalize blood glucose levels in a hypoglycemic host, 3) treat a host suffering from hypoglycemia, 4) treat a host suffering from nocturnal hypoglycemia, 5) treat a host suffering from hypoglycemia as a result of the host's use of insulin, and/or insulin analogs, and/or other blood glucose lowering agents (e.g. metformin), or a combination of such agents, 6) treat a host suffering from nocturnal hypoglycemia as a result of the host's use of insulin, and/or insulin analogs, and/or other blood glucose lowering agents (e.g. metformin), or a combination of such agents, 7) lessen the range of circadian blood glucose levels in a host, 8) treat a host suffering from idiopathic hypoglycemia, and 9) treat a host suffering from hypoglycemia associated with the use of prescription drugs.

This application also demonstrates that the inhibition of AMPA/kainate receptors can be used to: 1) decrease the secretion of glucagon in a host, 2) normalize blood glucose levels in a hyperglycemic host, 3) treat a host suffering from hyperglycemia, 4) treat a host suffering from daytime hyperglycemia, and 5) treat a host suffering from hyperglycemia associated with elevated levels of glucagon.

In some embodiments of the subject methods, inhibition rather than excitation of AMPA/kainate mediated responses is employed to counteract excessive daytime glucagon release in diabetic subjects in need. This is viewed as a complement to the excitation of AMPA/kainate-type glutamate receptors, whereby AMPA/kainate-type glutamate receptors can be alternately modulated over the circadian period to achieve enhanced or reduced glucagon secretion in diabetics as needed. Use of direct antagonist or negative modulators of AMPA-Rs to normalize daytime glucagon levels is provided in some instances of the subject methods.

Any convenient compounds that can act as antagonists and/or negative allosteric modulators of AMPA/kainite receptors can find use in certain embodiments of the subject methods. Non-limiting examples of chemical structures of compounds within the class of antagonists and negative allosteric modulators of AMPA/kainite receptors that find use in certain embodiments of the subject methods are set forth below. Antagonist compounds of interest include, but are not limited to, certain kainite or AMPA receptor antagonists as disclosed in U.S. Pat. Nos. 10,100,045, 9,469,632, 6,600,036, 6,160,133, 6,191,117, and 6,136,812, which are incorporated herein by reference.

In some instances, the antagonists and/or negative allosteric modulator compound is a quinoxaline derivative. Quinoxaline derivatives of interest include, but are not limited to, CNQX (see e.g., Qin et al. Brain Res. 2008 Sep. 4; 1228: 43-57), DNQX, NBQX, YM-90K, YM-872, other related derivatives. Exemplary quinoxalinedione derivatives are disclosed in De Sarro et al. (“AMPA receptor antagonists as potential anticonvulsant drugs. Curr Top Med Chem. 2005; 5(1):31-42), the disclosure of which is herein incorporated by reference. In certain embodiments, the antagonist and/or negative allosteric modulator compound is selected from one of the following structures:

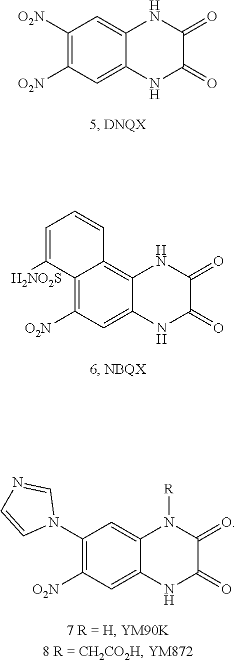

In some cases, the antagonist and/or negative allosteric modulator compound is a non-competitive antagonist (also known as “negative allosteric modulator”) of AMPA/kainite receptors selected from the class of 2,3-benzodiazepines and variants thereof, quinazolin-4-ones or and derivatives thereof, and quinoxalinediones, e.g., 1H-quinazoline-2,4-diones. Exemplary antagonist and/or negative allosteric modulator compounds of interest include, but are not limited to, GYKI 52466, talampanel (GYKI 53773; LY 300164) (see e.g., Erdo et al. “The AMPA-antagonist talampanel is neuroprotective in rodent models of focal cerebral ischemia,” Brain Res Bull. 2005 Jul. 15; 66(1):43-9), CP-465,022, perampanel (see e.g., Rogawski et al. “Preclinical pharmacology of perampanel, a selective non-competitive AMPA receptor antagonist,” Acta Neurol Scand Suppl. 2013; (197):19-24), caroverine (see e.g., Klement et al., “Blocking AMPA receptor signalling by caroverine infusion does not affect counter-regulation of hypoglycaemia in healthy men.”. Diabetologia. 2009 June; 52(6):1192-6), tezampanel (see e.g., Jin et al. “Epidural tezampanel, an AMPA/kainate receptor antagonist, produces postoperative analgesia in rats.” Anesth Analg. 2007 October; 105(4):1152-9), theanine (see e.g., Kakuda “Neuroprotective effects of theanine and its preventive effects on cognitive dysfunction.” Pharmacological Research 64 (2011) 162-168) and derivatives thereof. Exemplary compounds of interest, also include but are not limited to, those compounds disclosed by Madsen et al. (“Inhibitors of AMPA and Kainate Receptor,” Current Medicinal Chemistry, 8 (11), 2001, 1291-1301) and Rogawski M A. (“Revisiting AMPA receptors as an antiepileptic drug target.” Epilepsy Curr. 2011 March; 11(2):56-63), the disclosures of which are herein incorporated by reference. In certain embodiments, the antagonist and/or negative allosteric modulator compound is selected from one of the following structures:

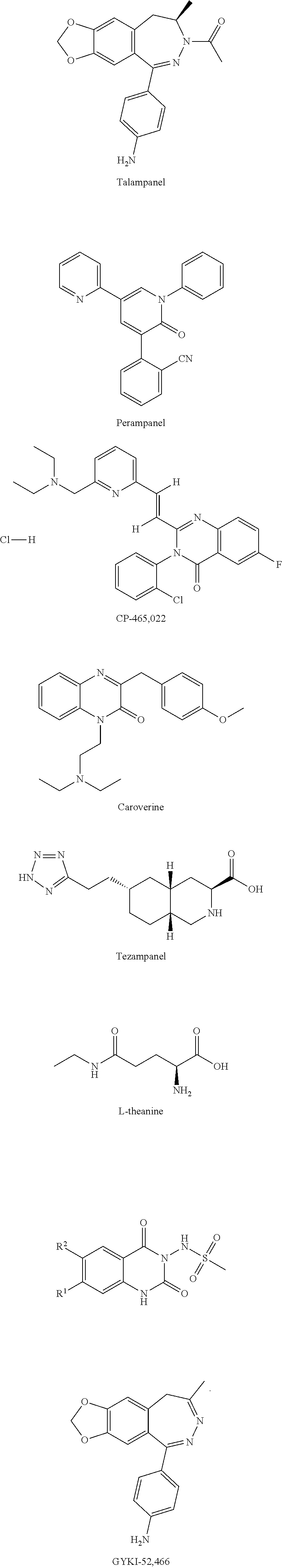

In certain instances, the antagonist and/or negative allosteric modulator compound is a quinazolin-4-one, see e.g., U.S. Pat. No. 6,136,812. In some embodiments, the antagonist quinazolin-4-one compound is a AMPA receptor antagonizing compound within group (A), (B), (C), (D), (E), or a pharmaceutically acceptable salt of said compound, wherein groups (A), (B), (C), (D), (E), and (F) are as follows:
(A) (S)-3-(2-chloro-phenyl)-2-[2-(5-diethylaminomethyl-2-fluoro-phenyl)-vinyl]-6-fluoro-3H-quinazolin-4-one;
(S)-3-(2-chloro-phenyl)-2-[2-(6-diethylaminomethyl-pyridin-2-yl)-vinyl]-6-fluoro-3H-quinazolin-4-one;
(S)-3-(2-chloro-phenyl)-2-[2-(4-diethylaminomethyl-pyridin-2-yl)-vinyl]-6-fluoro-3H-quinazolin-4-one;
(S)-3-(2-chloro-phenyl)-2-[2-(6-ethylaminomethyl-pyridin-2-yl)-vinyl]-6-fluoro-3H-quinazolin-4-one;
(S)-3-(2-bromo-phenyl)-2-[2-(6-diethylaminomethyl-pyridin-2-yl)-vinyl]-6-fluoro-3H-quinazolin-4-one;
(S)-3-(2-chloro-phenyl)-6-fluoro-2-[2-(6-methoxymethyl-pyridin-2-yl)-vinyl]-3H-quinazolin-4-one;
(S)-3-(2-chloro-phenyl)-6-fluoro-2-[2-(4-methyl-pyrimidine-2-yl)-vinyl]-3H-quinazolin-4-one;
(S)-3-(2-chloro-phenyl)-6-fluoro-2-{2-[6-isopropylamino-methyl)-pyridin-2-yl]-ethyl}-3H-quinazolin-4-one;
(S)-6-fluoro-2-[2-(2-methyl-thiazol-4-yl)-vinyl]-3-(2-methyl-phenyl)-3H-quinazolin-4-one;
(S)-3-(2-chloro-phenyl)-6-fluoro-2-[2-(2-methyl-thiazol-4-yl)-vinyl]-3H-quinazolin-4-one;
(S)-2-[2-(2-dimethylaminomethyl-thiazol-4-yl)-vinyl]-6-fluoro-3-(2-fluoro-phenyl)-3H-quinazolin-4-one;
(S)-3-(2-bromo-phenyl)-6-fluoro-2-[2-(2-methyl-thiazol-4-yl)-vinyl]-3H-quinazolin-4-one;
(S)-3-(2-chloro-phenyl)-2-[2-(2-methyl-thiazol-4-yl)-vinyl]-3H-quinazolin-4-one;
(S)-3-(2-chloro-phenyl)-6-fluoro-2-(2-pyridin-2-yl-vinyl)-3H-quinazolin-4-one;
(S)-3-(2-bromo-phenyl)-2-(2-pyridin-2-yl-vinyl)-3H-quinazolin-4-one;
(S)-6-chloro-2-(2-pyridin-2-yl-vinyl)-3-o-tolyl-3H-quinazolin-4-one;
(S)-3-(2-chloro-phenyl)-2-[2-(6-methyl-pyridin-2-yl)-vinyl]-3H-quinazolin-4-one;
(S)-6-chloro-2-[2-(6-methyl-pyridin-2-yl)-vinyl]-3-o-tolyl-3H-quinazolin-4-one;
(S)-3-(2-chloro-phenyl)-6-fluoro-2-(2-pyridin-2-yl-ethyl)-3H-quinazolin-4-one;
(S)-6-{2-[3-(2-chloro-phenyl)-6-fluoro-4-oxo-3,4-dihydro-quinazolin-2-yl]-vinyl}-pyridine-2-carbaldehyde;
(S)-3-(2-chloro-phenyl)-6-fluoro-2-[2-(6-methylaminomethyl-pyridin-2-yl)-vinyl]-3H-quinazolin-4-one;
(S)—N-(6-{2-(2-chloro-phenyl-6-fluoro-4-oxo-3,4-dihydro-quinazolin-2-yl]-vinyl}-pyridin-2-ylmethyl)-N-methyl-acetamide;
(S)-6-{2-[3-(2-chloro-phenyl)-6-fluoro-4-oxo-3,4-dihydro-quinazolin-2-yl]-vinyl}-pyridine-2-carbonitrile;
(S)-3-(2-fluoro-phenyl)-2-(2-pyridin-2-yl-vinyl)-3H-quinazolin-4-one;
(S)-3-(2-bromo-phenyl)-6-fluoro-2-(2-pyridin-2-yl-vinyl)-3H-quinazolin-4-one;
(S)-3-(2-chloro-phenyl)-2-[2-(6-diethylaminomethyl-pyridin-2-yl)-vinyl]-3H-quinazolin-4-one;
(S)—N-(6-{2-[3-(2-chloro-phenyl)-6-fluoro-4-oxo-3,4-dihydro-quinazolin-2-yl]-vinyl}-pyridin-2-ylmethyl)-N-ethyl-acetamide;
(S)-3-(2-chloro-phenyl)-6-fluoro-2-[2-(6-fluoromethyl-pyridin-2-yl)-vinyl]-3H-quinazolin-4-one;
(S)-3-(2-chloro-phenyl)-6-fluoro-[2-(6-pyrrolidin-1-ylmethyl-pyridin-2-yl)-ethyl]-3H-quinazolin-4-one;
(S)-3-(2-chloro-phenyl)-2-[2-(6-{(ethyl-(2-hydroxy-ethyl)-amino]-methyl}-pyridin-2-yl)-vinyl]-6-fluoro-3H-quinazolin-4-one;
(S)-3-(2-chloro-phenyl)-6-fluoro-2-{2-[6-(isopropylamino-methyl)-pyridin-2-yl]-vinyl}-3H-quinazolin-4-one;
(S)-3-(2-chloro-phenyl)-6-fluoro-2-{2-[6-(2-methyl-piperidin-1-ylmethyl)-pyridin-2-yl]-vinyl-4-one;
(S)-3-(2-chloro-phenyl)-2-[2-(6-ethoxymethyl-pyridin-2-yl)-vinyl]-6-fluoro-3H-quinazolin-4-one;
(S)-3-(2-chloro-phenyl)-2-{2-[6-(2,5-dihydro-pyrrol-1-ylmethyl)-pyridin-2-yl]-vinyl}-6-3H-quinazolin-4-one;
(S)-3-(2-chloro-phenyl)-6-fluoro-2-{2-[6-(4-methyl-piperidin-1-ylmethyl)-pyridin-2-yl]-vin-3H-quinazolin-4-one;
(S)-6-bromo-2-[2-(6-methyl-pyridin-2-yl)-vinyl]-3-o-tolyl-3H-quinazolin-4-one;
(S)-6-bromo-2-(2-pyridin-2-yl-vinyl)-3-o-tolyl-3H-quinazolin-4-one;
(S)-6-fluoro-3-(2-fluoro-phenyl)-2-(2-pyridin-2-yl-vinyl)-3H-quinazolin-4-one;
(S)-3-(2-chloro-phenyl)-6-methyl-2-(2-pyridin-2-yl-vinyl)-3H-quinazolin-4-one;
(S)-3-(2-chloro-phenyl)-2-[2-(6-dimethylaminomethyl-pyridin-2-yl)-vinyl]-6-fluoro-3H-quinazolin-4-one;
(S)-6-fluoro-3-(2-fluoro-phenyl)-2-[2-(6-methyl-pyridin-2-yl)-vinyl]-3H-quinazolin-4-one;
(S)-3-(2-chloro-phenyl)-2-[2-(6-{(2-dimethylamino-ethyl)-methyl-amino]-methyl}-pyridin-2-yl)-vinyl]-6-fluoro-3H-quinazolin-4-one;
(S)-3-(2-chloro-phenyl)-6-2-[2-(6-hydroxymethyl-pyridin-2-yl)-vinyl]-3H-quinazolin-4-one;
(S)-acetic acid 6-{2-[3-(2-chloro-phenyl)-6-fluoro-4-oxo-3,4-dihydro-quinazolin-2-yl]-vinyl}-pyridin-2-yl methyl ester;
(S)-6-{2-[3-(2-bromo-phenyl)-6-fluoro-4-oxo-3,4-dihydro-quinazolin-2-yl]-vinyl}-pyridine-2-carbaldehyde;
(S)-3-(2-bromo-phenyl)-2-[2-(6-diethylaminomethyl-pyridin-2-yl)-vinyl]-3H-q uinazolin-4-one;
(S)-acetic acid 6-{2-[3-(2-bromo-phenyl)-6-fluoro-4-oxo-3,4-dihydroquinazolin-2-yl]-vinyl}-pyridin-2-ylmethyl ester;
(S)-diethylamino-acetic acid 6-{2-[3-(2-chloro-phenyl)-6-fluoro-4-oxo-3,4-dihydro-quinazolin-2-yl]-vinyl}-pyridin-2-ylmethyl ester;
(S)-3-(2-chloro-phenyl)-2-[2-(6-difluoromethyl-pyridin-2-yl)-vinyl]-6-fluoro-3H-quinzaolin-4-one;
(S)-3-(2-chloro-phenyl)-2-[2-(6-methoxy-pyridin-2-yl)-vinyl]-3H-quinazolin-4-one;
(S)-2-{2-[3-(2-chloro-phenyl)-6-fluoro-4-oxo-3,4-dihydro-quinazolin-2-yl]-vinyl}-6-methyl-nicotinonitrile;
(S)-2-{2-[3-(2-chloro-phenyl)-6-fluoro-4-oxo-3,4-dihydro-quinazolin-2-yl]-ethyl}-6-methyl-nicotinonitrile;
(S)-3-(2-chloro-phenyl)-6-fluoro-2-(2-pyrimidine-2-yl-ethyl)-3H-quinazolin-4-one;
(S)-3-(2-chloro-phenyl)-2-[2-(4,6-dimethyl-pyrimidine-2-yl)-vinyl]-6-fluoro-3H-quinazolin-4-one;
(S)-2-{2-[3-(2-chloro-phenyl)-6-fluoro-4-oxo-3,4-dihydro-quinazolin-2-yl]-vinyl}-nicotinonitrile;
(S)-3-(2-chloro-phenyl)-6-fluoro-2-(2-{6-[(3-methyl-butylamino)-methyl]-pyridin-2-yl}-ethyl)-3H-quinazolin-4-one;
(S)-2-{2-[3-(2-chloro-phenyl)-6-fluoro-4-oxo-3,4-dihydro-quinazolin-2-yl]-ethyl}-nicotinonitrile;
(S)-2-[2-(6-chloro-4-oxo-3-o-tolyl-3,4-dihydro-quinazolin-2-yl)-vinyl]-benzonitrile;
(S)-2-{2-[3-(2-chloro-phenyl)-6-fluoro-4-oxo-3,4-dihydro-quinazolin-2-yl]-vinyl}-4-methyl-benzonitrile;
(S)-3-(2-bromo-phenyl)-6-fluoro-2-[2-(6-hydroxymethyl-pyridin-2-yl)-vinyl]-3H-quinazolin-4-one; and
(S)-3-(2-chloro-phenyl)-6-fluoro-2-[2-(6-pyrrolidin-1-ylmethyl-pyridin-2-yl-vinyl]-3H-quinazolin-4-one;
(B) (S)-6-fluoro-2-[2-(2-fluoro-phenyl)-vinyl]-3-(2-methyl-pyridin-3-yl)-3H-quinazolin-4-one;
(S)-2-{2-[6-fluoro-3-(2-methyl-pyridin-3-yl)-4-oxo-3,4-dihydro-quinazolin-2-yl]-vinyl}-benzonitrile;
(S)-2-{2-[6-fluoro-3-(2-methylpyridin-3-yl)-4-oxo-3,4-dihydroquinazolin-2-yl]-vinyl}-benzonitrile;
(S)-2-{2-[3-(2-chloro-pyridin-3-yl)-6-fluoro-4-oxo-3,4-dihydroquinazolin-2-yl]-vinyl}-benzonitrile;
(S)-2-{2-[6-fluoro-3-(2-methyl-pyridin-3-yl)-4-oxo-3,4-dihydro-quinazolin-2-yl]-vinyl}-4-methyl-benzonitrile;
(S)-2-{2-[3-(2-methyl-pyridin-3-yl)-4-oxo-3,4-dihydro-quinazolin-2-yl]-vinyl}-benzonitrile;
(S)-6-fluoro-3-(2-methyl-pyridin-3-yl)-2-[2-(thiazol-2-yl)-vinyl]-3H-quinazolin-4-one;
(S)-6-fluoro-3-(2-methyl-pyridin-3-yl)-2-[2-(2-methyl-thiazol-4-yl)-vinyl]-3H-quinazolin-4-one;
(S)-6-fluoro-3-(2-methyl-pyridin-3-yl)-2-[2-(4-methyl-thiazol-2-yl)-vinyl]-3H-quinazolin-4-one;
(S)-2-[2-(5-diethylaminomethyl-2-fluoro-phenyl)-vinyl]-6-fluoro-3-(2-methyl-pyridin-3-yl)-3H-quinazolin-4-one;
(S)-6-fluoro-2-[2-(2-fluoro-5-pyrrolidin-1-ylmethyl-phenyl)-vinyl]-3-(2-methyl-pyridin-3-yl)-3H-quinazolin-4-one;
(S)-3-(2-chloro-pyridin-3yl)-2-[2-(2-fluoro-phenyl)-vinyl]-3H-quinazolin-4-one;
(S)-3-(2-chloro-pyridin-3-yl)-6-fluoro-2-[2-(6-methyl-phenyl-2-yl)-vinyl]-3H-quinazolin-4-one;
(S)-3-(2-chloro-pyridin-3-yl)-6-fluoro-2-[2-(fluoro-phenyl)-vinyl]-3H-quinazolin-4-one;
(S)-6-chloro-2-[2-(2-fluoro-phenyl)-vinyl]-3-(2-methyl-pyridin-3-yl)-3H-quinazolin-4-one;
(S)-6-chloro-2-[2-(2-fluoro-phenyl)-vinyl]-3-(3-methyl-1-oxy-pyridin-4-yl)-3H-quinazolin-4-one;
(S)-3-{2-(3-(2-chloro-pyridin-3-yl)-6-fluoro-4-oxo-3,4-dihydro-quinazolin-2-yl]-vinyl}-benzaldehyde;
(S)-3-{2-[3-(2-chloro-pyridin-3-yl)-4-oxo-3,4-dihydro-quinazolin-2-yl]-vinyl}-benzaldehyde;
(S)-3-(2-chloro-pyridin-3-yl)-6-fluoro-2-[2-(3-hydroxymethyl-phenyl)-vinyl]-3H-quinazolin-4-one;
(S)-3-(2-chloro-pyridin-3-yl)-2-{2-[3(1,4-dioxa-8-aza-spiro[4.5]dec-8-ylmethyl)-phenyl]-vinyl}-6-fluoro-3-3H-quinazolin-4-one;
(S)-3-(2-chloro-pyridin-3-yl)-6-fluoro-2-{2-[3-(4-pyrrolidin-1-yl-piperidin-1-ylmethyl)-phenyl]-vinyl}-3H-quinazolin-4-one;
(S)-2-{2-[3-(2-chloro-pyridin-3-yl-6-fluoro-4-oxo-3,4-dihydro-quinazolin-2-yl]-vinyl}-benzonitrile;
(S)-2-{2-[3-(2-chloro-pyridin-3-yl)-4-oxo-3,4-dihydro-quinazolin-2-yl]-vinyl}-benzonitrile;
(S)-2-[2-(2-fluoro-phenyl)-vinyl]-3-(2-methyl-pyridin-3-yl)-3H-quinazolin-4-one;
(S)-3-(2-chloro-pyridin-3-yl)-6-fluoro-2-[2-hydroxy-phenyl)-vinyl]-3H-quinazolin-4-one;
(S)-6-fluoro-3-(2-methyl-pyridin-3-yl)-2-[2-(2-methyl-thiazol-4-yl)-ethyl]-3H-quinazolin-4-one;
(S)-6-fluoro-3-(2-chloro-pyridin-3-yl)-2-[2-(2-dimethylamino-methylthiazol-4-yl)-vinyl]-3H-quinazolin-4-one;
(S)-2-[2-(5-Diethylaminomethyl-2-fluoro-phenyl)-vinyl]-6-fluoro-3-(4-methyl-pyridin-3-yl)-3H-quinazolin-4-one;
(S)-4-Diethylaminomethyl-2-{2-[6-fluoro-3-(4-methyl-pyridin-3-yl)-4-oxo-3,4-dihydro-quinazolin-2-yl]-vinyl}-benzonitrile;
(S)-2-[2-(5-Diethylaminomethyl-2-fluoro-phenyl)-vinyl]-6-fluoro-3-(3-methyl-pyrazin-2-yl)-3H-quinazolin-4-one;
(S)-6-fluoro-3-(2-methyl-pyridin-3-yl)-2-[2-(2-dimethylamino-methylthiazol-4-yl)-vinyl]-3H-quinazolin-4-one;
(S)-6-fluoro-3-(2-methyl-pyridin-3-yl)-2-[2-(2-methyl-oxazol-4-yl)-vinyl]-3H-quinazolin-4-one;
(S)-6-fluoro-3-(2-chloro-pyridin-3-yl)-2-[2-(thiazol-2-yl)-vinyl]-3H-quinazolin-4-one;
(S)-6-fluoro-3-(4-methyl-pyridin-3-yl)-2-[2-(4-methyl-thiazol-2-yl)-vinyl]-3H-quinazolin-4-one;
(S)-3-(2-chloro-pyridin-3-yl)-6-fluoro-2-[2-(2-hydroxy-phenyl)-vinyl]-3H-quinazolin-4-one; and
(S)-6-fluoro-2-[2-(2-fluoro-5-pyrrolidin-1-ylmethyl-phenyl)-ethyl]-3-(2-methyl-pyridin-3-yl)-3H-quinazolin-4-one;
(C) 3-(2-chloro-phenyl)-6-fluoro-2-(2-pyridin-2-yl-vinyl)-3H-quinazolin-4-one;
3-(2-bromo-phenyl)-2-(2-pyridin-2-yl)-3H-quinazolin-4-one;
6-chloro-2-(2-pyridin-2-yl-vinyl)-3-o-tolyl-3H-quinazolin-4-one;
3-(2-chloro-phenyl)-2-[2-(6-methyl-pyridin-2-yl)-vinyl]-3H-quinazolin-4-one;
6-chloro-2-[2-(6-methyl-pyridin-2-yl)-vinyl]-3-o-tolyl-3H-quinazolin-4-one;
3-(2-chloro-phenyl)-6-fluoro-2-(2-pyridin-2-yl-ethyl)-3H-quinazolin-4-one;
6-{2-[3-(2-chloro-phenyl)-6-fluoro-4-oxo-3,4-dihydro-quinazolin-2-yl]-vinyl}-pyridine-2-carbaldehyde;
3-(2-chloro-phenyl)-6-fluoro-2-[2-(6-methylaminomethyl-pyridin-2-yl)-vinyl]-3H-quinazolin-4-one;
N-(6-{2-[3-(2-chloro-phenyl)-6-fluoro-4-oxo-3,4-dihydro-quinazolin-2-yl]-vinyl}-pyridin-2-ylmethyl)-N-methyl-acetamide;
3-(2-chloro-phenyl)-2-[2-(4-diethylaminomethyl-pyridin-2-yl)-vinyl]-6-fluoro-3H-quinazolin-4-one;
6-{2-[3-(2-chloro-phenyl)-6-fluoro-4-oxo-3,4-dihydro-quinazolin-2-yl]-vinyl}-pyridine-2-carbonitrile;
3-(2-fluoro-phenyl)-2-(2-pyridin-2-yl-vinyl)-3H-quinazolin-4-one;
3-(2-bromo-phenyl)-6-fluoro-2-(2-pyridin-2-yl-vinyl)-3H-quinazolin-4-one;
3-(4-bromo-2-chloro-phenyl)-6-fluoro-2-(2-pyridin-2-yl-vinyl)-3H-quinazolin-4-one;
3-(2-chloro-phenyl)-2-[2-(6-diethylaminomethyl-pyridin-2-yl)-vinyl)-3H-quinazolin-4-one;
N-(6-{2-[3-(2-chloro-phenyl)-6-fluoro-4-oxo-3,4-dihydro-quinazolin-2-yl]-vinyl}-pyridin-2-ylmethyl)-N-ethyl-acetamide;
3-(2-chloro-phenyl)-6-fluoro-2-[2-(6-fluoromethyl-pyridin-2-yl)-vinyl]-3H-quinazolin-4-one;
3-(2-chloro-phenyl)-6-fluoro-2-[2-(6-pyrrolidin-1-ylmethyl-pyridin-2-yl)-ethyl]-3H-quinazolin-4-one;
3-(2-chloro-phenyl)-2-[2-(6-{(ethyl-(2-hydroxy-ethyl)-amino]-methyl}-pyridin-2-yl)-vinyl]-6-fluoro-3H-quinazolin-4-one;
3-(2-chloro-phenyl)-6-fluoro-2-{2-[6-(isopropylamino-methyl)-pyridin-2-yl]-vinyl}-3H-quinazolin-4-one;
3-(2-chloro-phenyl)-6-fluoro-2-{2-[6-(2-methyl-piperidin-1-ylmethyl)-pyridin-2-yl]-vinyl}-3H-quinazolin-4-one;
3-(2-chloro-phenyl)-2-[2-(6-ethylaminomethyl-pyridin-2-yl)-vinyl]-6-fluoro-3H-quinazolin-4-one;
3-(2-chloro-phenyl)-2-[2-(6-ethoxymethyl-pyridin-2-yl)-vinyl]-6-fluoro-3H-quinazolin-4one;
3-(2-chloro-phenyl)-2-{2-[6-(2,5-dihydro-pyrrol-1-ylmethyl)-pyridin-2-yl]-vinyl}-6-fluoro-3H-quinazolin-4-one;
3-(2-chloro-phenyl)-6-fluoro-2-{2-[6-(4-methyl-piperidin-1-ylmethyl)-pyridin-2-yl]-vinyl}-3H-quinazolin-4-one;
6-bromo-2-[2-(6-methyl-pyridin-2-yl)-vinyl]-3-o-tolyl-3H-quinazolin-4-one;
6-bromo-2-(2-pyridin-2-yl-vinyl)-3-o-tolyl-3H-quinazolin-4-one;
6-fluoro-3-(2-fluoro-phenyl)-2-(2-pyridin-2-yl-vinyl)-3H-quinazolin-4-one;
1-benzyl-5-(2-methyl-[1,3]dioxolan-2-yl)-2-oxo-2,3-dihydro-1H-indole-3-carboxylic acid (3-phenylcarbamoyl-phenyl)-amide;
3-(2-chloro-phenyl)-6-methyl-2-(2-pyridin-2-yl-vinyl)-3H-quinazolin-4-one;
3-(2-chloro-phenyl)-2-[2-(6-dimethylaminomethyl-pyridin-2-yl)-vinyl]-6-fluoro-3H-quinazolin-4-one;
6-fluoro-3-(2-fluoro-phenyl)-2-[2-(6-methyl-pyridin-2-yl)-vinyl]-3H-quinazolin-4-one;
3-(2-chloro-phenyl)-2-[2-(6-{[(2-dimethylamino-ethyl)-methyl-amino]-methyl}-pyridin-2-yl)-vinyl]-6-fluoro-3H-quinazolin-4-one;
3-(2-chloro-phenyl)-6-fluoro-2-[2-(6-hydroxymethyl-pyridin-2-yl)-vinyl]-3H-quinazolin-4-one;
acetic acid 6-{2-[3-(2-chloro-phenyl)-6-fluoro-4-oxo-3,4-dihydro-quinazolin-2-yl]-vinyl}-pyridin-2-ylmethyl ester;
6-{2-[3-(2-bromo-phenyl)-6-fluoro-4-oxo-3,4-dihydro-quinazolin-2-yl]-vinyl}-pyridine-2-carbaldehyde;
3-(2-bromo-phenyl)-2-[2-(6-diethylaminomethyl-pyridin-2-yl)-vinyl]-6-fluoro-3H-quinazolin-4-one;
3-(2-bromo-phenyl)-2-[2-(6-diethylaminomethyl-pyridin-2-yl)-vinyl]-3H-quinazolin-4-one;
acetic acid 6-{2-[3-(2-bromo-phenyl)-6-fluoro-4-oxo-3,4-dihydro-quinazolin-2-yl]-vinyl }-pyridin-2-ylmethyl ester;
3-(2-chloro-phenyl)-6-fluoro-2-[2-(6-methoxymethyl-pyridin-2-yl)-vinyl]-3H-quinolin-4-one;
diethylamino-acetic acid 6-{2-[3-(2-chloro-phenyl)-6-fluoro-4-oxo-3,4-dihydro-quinazolin-2-yl]-vinyl}-pyridin-2-ylmethyl ester;
6-fluoro-3-(2-methyl-pyridin-3-yl)-2-[2-(2-methyl-thiazol-4-yl)-vinyl]-3H-quinazolin-4-one;
3-(2-bromo-phenyl)-6-fluoro-2-[2-(6-hydroxymethyl-pyridin-2-yl)vinyl]-3H-quinazolin-4-one; and,
3-(2-chloro-phenyl)-6-fluoro-2-[2-(6-pyrrolidin-1-ylmethyl-pyridin-2-yl)-vinyl]-3H-quinazolin-4-one;
(D) 6-Chloro-3-(2-chloro-phenyl)-2-[2-hydroxy-2-(6-methyl-pyridin-2-yl)-vinyl]-3H-quinazolin-4-one; 2-{2-[3-(2-Chloro-phenyl)-4-oxo-3,4-dihydro-quinazolin-2-yl]-1-hydroxy-vin yl}-nicotinonitrile;
2-{2-[3-(2-Chloro-pyrid-3-yl)-6-fluoro-4-oxo-3,4-dihydro-quinazolin-2-yl]-1-hydroxy-vinyl}-nicotinonitrile;
2-{2-[6-Chloro-3-(2-methyl-phenyl)-4-oxo-3,4-dihydro-quinazolin-2-yl]-1-hydroxy-vinyl]-nicotinonitrile;
3-(2-Chloro-phenyl)-2-[2-(3-diethylaminomethyl-phenyl)-2-hydroxy-ethoxy]-6-fluoro-3H-quinazolin-4-one;
3-(2-Chloro-phenyl)-6-fluoro-2-[2-(3-pyrrolidin-1-ylmethyl-phenyl)-2-hydroxy-ethyl]-3H-quinazolin-4-one;
3-(2-Chloro-pyrid-3-yl)-2-[2-(3-diethylaminomethyl-phenyl)-2-hydroxy-ethyl]-6-fluoro-3H-quinazolin-4-one;
2-[2-(3-Diethylaminomethyl-phenyl)-2-hydroxy-ethyl]-6-fluoro-3-(2-fluoro-phenyl)-3H-quinazolin-4-one;
2-[2-(3-Diethylaminomethyl-phenyl)-2-hydroxy-ethyl]-3-(2-fluoro-phenyl)-3H-quinazolin-4-one;
3-(2-chloro-phenyl)-2-[2-(6-diethylaminomethyl)-pyridin-2-yl)-2-hydroxy-vinyl]-6-fluoro-3H-quinazolin-4-one;
2-[2-[3-(2-Chloro-pyrid-3-yl)-6-fluoro-4-oxo-3,4-dihydro-quinazolin-2-yl]-1-hydroxy-vinyl}-6-methyl-nicotinonitrile;
2-{2-[3-(2-Chloro-phenyl)-4-oxo-3,4-dihydro-quinazolin-2-yl]-1-hydroxy-vinyl)-6-methyl-nicotinonitrile;
2-{2-[6-Chloro-3-(2-chloro-phenyl)-4-oxo-3,4-dihydro-quinazolin-2-yl]-1-hydroxy-vinyl}-6-methyl-nicotinonitrile;
2-{2-[3-(2-Chloro-phenyl)-6-fluoro-4-oxo-3,4-dihydro-quinazolin-2-yl]-1-hydroxy-vinyl}-6-fluoro-nicotinonitrile;
2-{2-[3-(2-Chloro-phenyl)-6-fluoro-4-oxo-3,4-dihydro-quinazolin-2-yl]-1-hydroxy-vinyl}-4-fluoro-benzonitrile;
2-{2-[3-(2-Chloro-phenyl)-6-fluoro-4-oxo-3,4-dihydro-quinazolin-2-yl]-1-hydroxy-vinyl}-4-methyl-benzonitrile;
2-{2-[3-(2-Chloro-phenyl)-4-oxo-3,4-dihydro-thieno[3,2d]pyrimidin-2-yl]-1-hydroxy-vinyl}-6-methyl-nicotinonitrile;
2-{2-[3-(2-methyl-phenyl)-4-oxo-3,4-dihydro-thieno[3,2d]pyrimidin-2-yl]-1-hydroxy-vinyl}-6-methyl-nicotinonitrile;
2-{2-[3-(2-Chloro-pyrid-3yl)-4-3,4-dihydro-thieno[3,2-d]pyrimidin-2-yl]-1-hydroxy-vinyl}-4-methyl-benzonitrile;
2-{2-[3-(2-Chloro-phenyl)-4-oxo-3,4-dihydro-thieno[3,2-d]pyrimidin-2-yl]-1-hydroxy-vinyl}-4-fluoro-benzonitrile;
2-{2-[3-(2-Fluoro-phenyl)-4-oxo-3,4-dihydro-thieno[3,2-d]pyrimidin-2-yl]-1-hydroxy-vinyl}-4-methyl-benzonitrile;
2-{2-[3-(2-Chloro-phenyl)-4-oxo-3,4-dihydro-thieno[3,2-d]pyrimidin-2-yl]-1-hydroxy-vinyl}-benzonitrile; and,
2-{2-[3-(2-Chloro-pyrid-3yl)-4-oxo-3,4-dihydro-thieno[3,2-d]pyrimidin-2-yl]-1-hydroxy-vinyl}-benzonitrile;
3-(2-chloro-phenyl)-6-fluoro-2-[2-hydroxy-2-(2-methyl-thiazol-4-yl)-vinyl]-3H-quinazolin-4-one;
3-(2-chloro-phenyl)-6-fluoro-2-[2-hydroxy-2-(6-methyl-pyridin-2-yl)-vinyl]-3H-quinazolin-4-one;
2-{2-[3-(2-chloro-phenyl)-6-fluoro-4-oxo-3,4-dihydro-quinazolin-2-yl]-1-hydroxy-vinyl}-6-methyl-nicotinonitrile;
2-{2-[3-(2-chloro-phenyl)-6-fluoro-4-oxo-3,4-dihydro-quinazolin-2-yl]-1-hydroxy-vinyl}-nicotinonitrile;
2-{2-[3-(2-chloro-phenyl)-6-fluoro-4-oxo-3,4-dihydro-quinazolin-2-yl]-1-hydroxy-vinyl}-benzonitrile;
2-{2-[3-(2-chloro-pyridin-3-yl)-6-fluoro-4-oxo-3,4-dihydro-quinazolin-2-yl]-1-hydroxy-vinyl}-6-methyl-nicotinonitrile;
3-(2-chloro-phenyl)-6-fluoro-2-(2-hydroxy-2-pyridin-2-yl-vinyl)-3H-quinazolin-4-one;
2-{2-[6-fluoro-3-(2-methyl-pyridin-3-yl)-4-oxo-3,4-dihydro-quinazolin-2-yl]-1-hydroxy-vinyl}-benzonitrile;
2-{2-[3-(2-chloro-pyridin-3-yl)-6-fluoro-4-oxo-3,4-dihydro-quinazolin-2-yl]-1-hydroxy-vinyl}-benzonitrile;
3-(2-chloro-phenyl)-6-fluoro-2-[2-(2-fluoro-phenyl)-2-hydroxy-ethyl]-3H-quinazolin-4-one;
(E) 3-(2-chloro-phenyl)-6-fluoro-2-[(pyridin-2-ylmethyl)-amino]-3H-quinazolin-4-one;
6-fluoro-3-(2-methyl-phenyl)-2-[(pyridin-2-ylmethyl)-amino]-3H-quinazolin-4-one;
3-(2-chloro-phenyl)-6-fluoro-2-[(2-fluorophenyl-methyl)-amino]-3H-quinazolin-4-one;
3-(2-chloro-phenyl)-2-[(2-cyanophenyl-methyl)-amino]-6-fluoro-3H-quinazolin-4-one;
3-(2-chloro-phenyl)-2-[(6-diethylaminomethylpyridin-2-ylmethyl)-amino]-6-fluoro-3H-quinazolin-4-one;
3-(2-chloro-phenyl)-6-fluoro-2-[(6-pyrrolidin-1-ylmethyl-pyridin-2-ylmethyl)-amino]-3H-quinazolin-4-one;
3-(2-chloro-phenyl)-2-[(3-pyrrolidin-1-ylmethyl-phenylamino)-methyl]-3H-thieno[3,2-d]pyrimidin-4-one;
3-(2-methyl-phenyl)-2-[(3-pyrrolidin-1-ylmethyl-phenylamino)-methyl]-3H-thieno[3,2-d]pyrimidin-4-one;
3-(2-chloro-phenyl)-2-[(2-fluoro-phenylamino)-methyl]-3H-thieno[3,2d]pyrimidin-4-one;
3-(2-chloro-pyrid-3-yl)-2-[(3-pyrrolidin-1-ylmethyl-phenylamino)-methyl]-3H-thieno[3,2-d]pyrimidin-4-one;
2-{[3-(2-chloro-pyrid-3-yl)-4-oxo-3,4-dihydro-thieno[3,2-d]pyrimidin-2-ylmethyl]-amino}-benzonitrile;
3(2-chloro-phenyl)-2-[(3-pyrrolidin-1-ylmethyl-phenylamino)-methyl]-3H-quinazolin-4-one;
6-chloro-3-(2-chloro-phenyl)-2-[(3-pyrrolidin-1-ylmethyl-phenylamino)-methyl]-3H-quinazolin-4-one;
6-chloro-3-(2-chloro-phenyl)-2-[(3-diethylaminomethyl-phenylamino)-methyl]-3H-quinazolin-4-one;
6-chloro-3-(2-chloro-pyrid-3-yl)-2-[(3-diethylaminomethyl-phenylamino)-methyl]-3H-quinazolin-4-one;
6-chloro-3-(2-trifluoromethyl-phenyl)-2-[(3-diethylaminomethyl-phenylamino)-methyl]-3H-quinazolin-4-one;
2-{[3-(2-chloro-pyridin-3-yl)-4-oxo-3,4-dihydro-quinazolin-2-ylmethyl]-amino}-benzonitrile;
2-{[3-(2-methyl-pyridin-3-yl)-4-oxo-3,4-dihydro-quinazolin-2-ylmethyl]-amino}-benzonitrile;
2-{[6-fluoro-3-(2-methyl-phenyl)-4-oxo-3,4-dihydro-quinazolin-2-ylmethyl]-amino}-nicotinonitrile;
2-{[3-(2-chloro-phenyl)-4-oxo-3,4-dihydro-quinazolin-2-ylmethyl]-amino}-nicotinonitrile;
2-{[3-(2-chloro-pyridin-3-yl)-6-fluoro-4-oxo-3,4-dihydro-quinazolin-2-ylmethyl]-amino}-benzonitrile;
3-{[3-(2-chloro-phenyl)-6-fluoro-4-oxo-3,4-dihydro-quinazolin-2-ylmethyl]-amino}-benzonitrile;
3-(2-chloro-phenyl)-2-[(3-diethylaminomethyl-phenylamino)-methyl]-6-fluoro-3H-quinazolin-4-one;
3-(2-chloro-phenyl)-6-fluoro-2-(pyrimidin-2-ylaminomethyl)-3H-quinazolin-4-one;
3-(2-chloro-pyridin-3-yl)-6-fluoro-2-(m-tolylamino-methyl)-3H-quinazolin-4-one;
3-(2-chloro-pyridin-3-yl)-6-fluoro-2-[(6-methyl-pyridin-2-ylamino)-methyl]-3H-quinazolin-4-one;
3-(2-chloro-phenyl)-6-fluoro-2-(pyridin-2-ylaminomethyl)-3H-quinazolin-4-one;
3-(2-chloro-pyridin-3-yl)-6-fluoro-2-[(3-pyrrolidin-1-ylmethyl-phenylamino)-methyl]-3H-quinazolin-4-one;
6-fluoro-3-(2-methyl-pyridin-3-yl)-2-[(3-pyrrolidin-1-ylmethyl-phenylamino)-methyl]-3H-quinazolin-4-one;
3-(2-chloro-phenyl)-6-fluoro-2-[(2-fluoro-benzylamino)-methyl]-3H-quinazolin-4-one;
N-(3-{[3-3-(2-chloro-phenyl)-6-fluoro-4-oxo-3,4-dihydro-quinazolin-2-ylmethyl]-amino}-phenyl)-acetamide;
3-(2-chloro-phenyl)-6-fluoro-2-[(3-pyrrolidin-1-ylmethyl-phenylamino)-methyl]-3H-quinazolin-4-one;
2-{[3-(2-chloro-phenyl)-6-fluoro-4-oxo-3,4-dihydro-quinazolin-2-ylmethyl]-amino}-nicotinonitrile;
3-(2-chloro-pyridin-3-yl)-6-fluoro-2-[(2-fluoro-phenylamino)-methyl]-3H-quinazolin-4-one;
3-(2-chloro-phenyl)-6-fluoro-2-[(2-fluoro-phenylamino)-methyl]-3H-quinazolin-4-one; and
3-(2-chloro-phenyl)-6-fluoro-2-[(6-methyl-pyridin-2-ylamino)-methyl]-3H-quinazolin-4-one.

### Systems and Kits

Also provided are kits that include the subject compounds (e.g., as described herein). In some cases, the subject compound is a positive allosteric modulator of AMPA/kainite receptors. In some cases, the subject compound is an antagonist and/or negative allosteric modulator of AMPA/kainite receptors. Systems of the present disclosure include collections of active agents brought together, e.g., by a health care practitioner, for administration to a subject, such as a patient. Such systems may include a subject compound and one or more additional active agents. Kits that include the subject compounds which are provided that may include one or more dosages of the compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat conditions of interest (e.g., as described herein). These instructions may be present in the subject systems and kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### REFERENCES

References regarding the varied chemical structures that comprise the class of compounds known in the art as “ampakines” or “positive modulators of AMPA/kainate receptors”, and other similar terms, can be found in Trends Neurosci. 2006 October; 29(10):554-62; Cuff Opin Pharmacol. 2004 February; 4(1):4-11; Drugs. 2000 January; 59(1):33-78; Psychopharmacology (Berl). 2005 April; 179(1):4-29; Cuff Opin Pharmacol. 2006 February; 6(1):82-8; Cuff Drug Targets CNS Neurol Disord. 2004 June; 3(3):181-94; CNS Drug Rev. 2002 Fall; 8(3):255-82; Neuropharmacology. 2001 June; 40(8):976-83; Neuropharmacology. 2001 June; 40(8):1003-9; Curr Drug Targets. 2007 May; 8(5):603-20; Bioorg Med Chem Lett. 2006 Oct. 1; 16(19):5057-61; and Curr Drug Targets CNS Neurol Disord. 2004 June; 3(3):181-94; Curr Top Med Chem. 2016; 16(29):3536-3565; ACS Med Chem Lett. 2015 Feb. 11; 6(4):392-6; Curr Opin Pharmacol. 2015 February; 20:46-53; Mol Pharmacol. 2017 June; 91(6):576-585. These are included here by reference, as are derivatives of classes defined therein.

References regarding diabetes, hypoglycemia, glucagon, the role of glutamate in the modulation of blood sugar levels, and the like can be found in: Clin Pract. 2004 September; 65 Suppl 1:S41-6; Cell Metab. 2008 June; 7(6):545-54; N Engl J Med., 1988 Nov. 10; 319(19):1233-9. PMID; and Clinical Diabetes July 2006 vol. 24 no. 3 115-121.

It is evident from the above results and discussion that novel methods to conveniently regulate the blood glucose levels, particularly the treatment of hypoglycemia, and especially hypoglycemia occurring as a result of standard diabetes medications, of a mammal are provided. Furthermore, since the subject methods modulate blood glucose levels through regulation of an autocrine step that elicits and potentiates endogenous glucagon production from pancreatic alpha cells, the possibility exists to achieve better regulation of blood glucose levels than is obtainable with conventional therapeutic regimes. One such conventional method is the consumption of high carbohydrate meals before going to sleep for the night. An emerging option is time-released glucagon. Diabetics, for instance, can employ each of these to offset hypoglycemia at night, but have the disadvantage of raising glucose each time they are employed, whether or not hypoglycemia would be manifest on a given night. The instant method would only raise glucagon and blood glucose levels when the body needs it.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

To demonstrate that positive allosteric modulators of AMPA-type glutamate receptors (PAMs) augment glucagon secretion conditionally during hypoglycemia, but not during euglycemia or hyperglycemia, in vitro and in vivo experimental systems are used. In an in vitro approach, isolated rodent islets maintained in culture in 96-well plates are treated for 1 hr with PAMs. Simultaneous with addition of PAMs, islets are exposed by media exchange to glucose concentrations ranging from hypoglycemic to hyperglycemic: e.g., 2 mM, 6 mM, 12 mM, 18 mM, etc. At the end of a 1 hour PAM treatment in varying glucose concentrations, glucagon levels in each treatment condition are then measured by ELISA.

Four distinct PAMs (aniracetam, LY404187, ORG26576, and GSK20) are observed to dose-dependently upregulate the amount of glucagon released into a culture media by isolated islets under hypoglycemic conditions, but not during euglycemic and hyperglycemic conditions. A vehicle control does not upregulate glucagon secretion at any glucose concentration.

### Example 2

In an in vivo approach, 2 rodent models of diabetes are utilized: high fat diet-induced obesity in mice (a model of Type II) and streptozotocin (STZ)-treated rats (a model of Type I). In each model, multiple episodes of hypoglycemia are first induced by repeated i.p. injection with human insulin over 3 days to exacerbate the impairment in hypoglycemia-induced glucagon secretion seen in these models (which models an effect seen in human diabetics during insulin use). On day 4, PAMs are administered (or vehicle control) by i.p. injection 30-90 min prior to challenging the rodents with a dose of human insulin sufficient to induce 3 hrs of hypoglycemia. Blood glucagon, insulin, and glucose (to confirm hypoglycemia) levels are measured in samples collected at, e.g., 10 min, 30 min, 60 min, 90 min, 120 min and 180 min relative to the time of insulin injection. For controls, the same studies are performed in the diabetes models without the insulin challenge on day 4, and in normal rodents during a single 3 hour episode of insulin-induced hypoglycemia.

In such studies, PAMs are observed to augment the level of glucagon secretion and glucose in vivo during hypoglycemic challenge in diabetic rodents, and to a lesser extent in normal rodents, but do not elevate glucagon or glucose during euglycemic or hyperglycemic states. Altogether, PAMs can offer a conditional enhancement of glucagon secretion in isolated islets and rodent models of diabetes. PAMs can be used as a prophylactic therapy to prevent nocturnal hypoglycemia in human Type I and Type II diabetics using insulin.

Notwithstanding the appended claims, the disclosure is also defined by the following clauses:

Clause 1. A method for increasing blood glucose levels in a mammalian host, the method comprising: administering to the host an effective amount of a positive allosteric modulator of an AMPA/kainate receptor (e.g., as described herein) to increase the blood glucose levels in the mammalian host.
Clause 2. The method according to clause 1, where the mammalian host is a human.
Clause 3. The method according to clause 1, wherein said administering is by oral delivery.
Clause 4. The method according to clause 1, wherein said administering occurs over a time period between one and twelve days.
Clause 5. The method according to clause 1, wherein said administering occurs over a time period greater than twelve days.
Clause 6. A method for increasing glucagon levels in a mammalian host, the method comprising: administering to the host an effective amount of a positive allosteric modulator of an AMPA/kainate receptor (e.g., as described herein) to increase the glucagon levels in the mammalian host.
Clause 7. The method according to clause 6, where the mammalian host is a human.
Clause 8. The method according to clause 6, wherein said administering is by oral delivery.
Clause 9. The method according to clause 6, wherein said administering occurs over a time period between one and twelve days.
Clause 10. The method according to clause 6, wherein said administering occurs over a time period greater than twelve days.
Clause 11. A method for increasing blood glucose levels in a mammalian host, the method comprising: administering to the host an effective amount of a positive allosteric modulator of an AMPA/kainate receptor (e.g., as described herein) of the pancreas to increase the blood glucose levels in the mammalian host.
Clause 12. The method according to clause 11, where the mammalian host is a human.
Clause 13. The method according to clause 11, wherein said administering is by oral delivery.
Clause 14. A method for treating hypoglycemia in a mammalian host, the method comprising: administering to the host an effective amount of a positive allosteric modulator of an AMPA/kainate receptor (e.g., as described herein) to increase the blood glucose levels in the mammalian host increases.
Clause 15. The method according to clause 14, wherein the mammalian host is suffering from nocturnal hypoglycemia.
Clause 16. The method according to clause 14, wherein the hypoglycemia is the result of a disease or disorder characterized by abnormally low blood levels of glucose.
Clause 17. The method according to clause 14, wherein the hypoglycemia is the result of the host taking insulin or an insulin analog or an antiglycemic agent, or any combination of such agents.
Clause 18. The method according to clause 14, wherein said administering is by oral delivery.
Clause 19. The method according to clause 14, where the mammalian host is a human.
Clause 20. The method according to clause 14, where the mammalian host is a human with diabetes.
Clause 21. A method for decreasing the size of the circadian range of blood glucose levels in a mammalian host, the method comprising: administering to the host an effective amount of a positive allosteric modulator of an AMPA/kainate receptor (e.g., as described herein) to decrease the size of the circadian range of blood glucose levels in the mammalian host.
Clause 22. The method according to clause 20, wherein the host has taken insulin or an insulin analog or another antiglycemic agent, or any combination of such agents.
Clause 23. The method according to clause 20, wherein said administering is by oral delivery.
Clause 24. The method according to clause 20, where the mammalian host is a human.
Clause 25. The method according to clause 20, where the mammalian host is a human with diabetes.
Clause 26. A method for enhancing the regulation of blood glucose levels of a human patient in need thereof by potentiating AMPA/kainate receptors, wherein the method comprises administration of a pharmaceutical composition able to potentiate the AMPA/kainate receptors, said administration in an amount sufficient to increase the size of AMPA/kainate receptor-mediated responses.
Clause 27. The method of clause 26 where the potentiated AMPA/kainate receptors are located in the pancreas of the patient.
Clause 28. The method of clause 26 where the potentiated AMPA/kainate receptors are located in the alpha-cells of the pancreas of the patient.
Clause 29. A method of increasing blood glucose levels in a human patient in need thereof by potentiating AMPA/kainate pancreas receptors wherein the method comprises administration of a pharmaceutical composition able to potentiate the AMPA/kainate receptors, said administration in an amount sufficient to increase the size of AMPA/kainate receptor-mediated responses.
Clause 30. The method of clause 29 where the potentiated AMPA/kainate receptors are located in the alpha-cells of the pancreas of the patient.
Clause 31. A method of treating hypoglycemia in a human patient in need thereof by potentiating AMPA/kainate receptors in the pancreas wherein the method comprises administration of a pharmaceutical composition able to potentiate the AMPA/kainate receptors, said administration in an amount sufficient to increase the size of AMPA/kainate receptor-mediated responses.
Clause 32. The method of clause 31 where the potentiated AMPA/kainate receptors are located in the alpha-cells of the pancreas of the patient.
Clause 33. The method of clause 31 where the hypoglycemia of the patient is nocturnal hyperglycemia.
Clause 34. A method for treating low blood glucose levels in a subject, said method comprising administering an effective amount of a composition that comprises a compound that enhances the stimulation of AMPA/kainate receptors (e.g., as described herein) in said subject.
Clause 35. The method of clause 34 wherein the composition is administered orally.
Clause 36. The method of clause 34 wherein the composition is administered by injection.
Clause 37. A kit, comprising a container containing the composition of clause 34 and instructions for using the composition for treating low blood glucose levels in a subject.
Clause 38. A method for treating hyperglycemia in a subject, said method comprising pharmacologically amplifying natural stimulators of AMPA/kainate receptors in said subject to enhance the mediation by said receptors of excitatory synaptic responses, said amplification being sufficient to reduce the symptoms of hyperglycemia.
Clause 39. A method in accordance with clause 38 comprising pharmacologically amplifying said natural stimulators by administering to said subject an effective amount of a compound selected from the group of compounds capable of increasing the activity of AMPA/kainate receptors (e.g., as described herein).
Clause 40. The method of clause 38 where the excitatory responses occur in the alpha-cells of the pancreas of the subject.
Clause 41. A method for treating idiopathic hypoglycemia in a mammalian host, the method comprising: administering to the host a positive allosteric modulator of an AMPA/kainate receptor (e.g., as described herein); whereby the blood glucose levels in the mammalian host increases.
Clause 42. The method according to clause 41, wherein said administering is by oral delivery.
Clause 43. The method according to clause 41, wherein said administering is by injection.
Clause 44. The method according to clause 41, where the mammalian host is a human.
Clause 45. A method for treating hypoglycemia associated with the use of prescription drug in a mammalian host, the method comprising: administering to the host a positive allosteric modulator of an AMPA/kainate receptor (e.g., as described herein); whereby the blood glucose levels in the mammalian host increases.
Clause 46. The method according to clause 45, wherein said administering is by oral delivery.
Clause 47. The method according to clause 45, wherein said administering is by injection.
Clause 48. The method according to clause 45, where the mammalian host is a human.
Clause 49. A method for decreasing blood glucagon levels in a mammalian host, the method comprising: administering to the host an antagonist or a negative allosteric modulator of a AMPA/kainate receptor (e.g., as described herein); whereby the blood glucagon levels in the mammalian host decreases.
Clause 50. The method according to clause 49, where the mammalian host is a human.
Clause 51. The method according to clause 49, wherein said administering is by oral delivery.
Clause 52. The method according to clause 49, wherein said administering occurs over a time period between one and twelve days.
Clause 53. The method according to clause 49, wherein said administering occurs over a time period greater than twelve days.
Clause 54. A method for decreasing blood glucose levels in a mammalian host, the method comprising: administering to the host an antagonist or a negative allosteric modulator of an AMPA/kainate receptor of the pancreas (e.g., as described herein): whereby the blood glucose levels in the mammalian host decreases.
Clause 55. The method according to clause 54, where the mammalian host is a human.
Clause 56. The method according to clause 54, wherein said administering is by oral delivery.
Clause 57. A method of decreasing blood glucagon levels in a human patient in need thereof by antagonizing AMPA/kainate pancreas receptors wherein the method comprises administration of a pharmaceutical composition able to inhibit AMPA/kainate receptors, said administration in an amount sufficient to decrease the size of AMPA/kainate receptor-mediated responses.
Clause 58. The method of clause 57 where the inhibited AMPA/kainate receptors are located in the alpha-cells of the pancreas of the patient.

Although the foregoing invention has been described in some detail by way of illustration and example for purposes of clarity of understanding, it will be readily apparent to those of ordinary skill in the art, in light of the teachings of this invention, that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. This includes the use of positive allosteric modulators of AMPA/kainate receptors that have not been cited as illustrations in this document.

Furthermore, all examples and conditional language recited herein are simply intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method for increasing blood glucose levels in a mammalian host, the method comprising:
administering to the host an effective amount of a positive allosteric modulator of an AMPA/kainate receptor to increase the blood glucose and/or glucagon levels in the mammalian host.

2. The method according to claim 1 wherein the mammalian host is suffering from diabetes Type I or Type II.

3. The method according to claim 1, wherein the method treats hypoglycemia or idiopathic hypoglycemia in the mammalian host.

4. The method according to claim 2, wherein the mammalian host is suffering from nocturnal hypoglycemia.

5. The method according to claim 2, wherein the hypoglycemia is the result of a disease or disorder characterized by abnormally low blood levels of glucose.

6. The method according to claim 2, wherein the hypoglycemia is the result of the host taking insulin or an insulin analog or an antiglycemic agent, or any combination of such agents.

7. A method for increasing blood glucose levels in a mammalian host, the method comprising:
administering to the host an effective amount of a positive allosteric modulator of an AMPA/kainate receptor of the pancreas to increase the blood glucose levels in the mammalian host.

8. A method for decreasing the size of the circadian range of blood glucose levels in a mammalian host, the method comprising:
administering to the host an effective amount of a positive allosteric modulator of an AMPA/kainate receptor to decrease the size of the circadian range of blood glucose levels in the mammalian host.

9. The method according to claim 8, wherein the host has taken insulin or an insulin analog or another antiglycemic agent, or any combination of such agents.

10. The method according to any one of claims 1-9, where the mammalian host is a human.

11. The method according to claim 10, where the mammalian host is a human with diabetes Type I or Type II.

12. The method according to any one of claims 1-11, wherein said administering is by oral delivery.

13. The method according to any one of claims 1-12, wherein said administering occurs:
a) intermittently; or
b) over a time period between one and twelve days.

14. The method according to any one of claims 1-13, wherein said administering occurs over a time period greater than twelve days.

15. A method for treating hyperglycemia in a subject, said method comprising pharmacologically amplifying natural stimulators of AMPA/kainate receptors in said subject to enhance the mediation by said receptors of excitatory synaptic responses, said amplification being sufficient to reduce the symptoms of hyperglycemia, the method comprising administering to said subject an effective amount of a compound selected from the group of compounds capable of increasing the activity of AMPA/kainate receptors.

16. The method of claim 15 where the excitatory responses occur in the alpha-cells of the pancreas of the subject.

17. A method for treating hypoglycemia associated with the use of prescription drug in a mammalian host, the method comprising: administering to the host a positive allosteric modulator of an AMPA/kainate receptor, whereby the blood glucose levels in the mammalian host increases.

18. A method for decreasing blood glucagon levels in a mammalian host, the method comprising: administering to the host an antagonist or a negative allosteric modulator of a AMPA/kainate receptor; whereby the blood glucose or blood glucagon levels in the mammalian host decreases.

19. The method of claim 18 where the inhibited AMPA/kainate receptors are located in the alpha-cells of the pancreas of the patient.

20. A kit, comprising a container containing an effective dose of a composition comprising a compound that enhances the stimulation of AMPA/kainate receptors (e.g., as described herein); and instructions for using the composition for treating low blood glucose levels in a subject.
